(19) United States
(12) Patent Application Publication
(10) Pub. No.: US 2021/0024974 A1
(43) Pub. Date: Jan. 28, 2021

(54) High-Throughput Cell-Based Screening Methodology For Evaluating Carbohydrate-Active Enzymes

(71) Applicant: Rutgers, the State University of New Jersey, New Brunswick, NJ (US)

(72) Inventors: Ayushi AGRAWAL, New Brunswick, NJ (US); Chandra Kanth Bandi, New Brunswick, NJ (US); Shishir Chundawat, Robbinsville, NJ (US)

(21) Appl. No.: 16/935,286

(22) Filed: Jul. 22, 2020

Related U.S. Application Data
(60) Provisional application No. 62/877,021, filed on Jul. 22, 2019.

(51) Int. Cl. C12Q 1/26; G01N 33/68

(52) U.S. Cl. CPC C12Q 1/26; G01N 2333/90245; G01N 33/6872

(57) ABSTRACT

The present disclosure relates, in one aspect, to the discovery of a high throughput screening (HTS) method to rapidly screen for GH/GS variants that are generated using directed evolution techniques and that can significantly enhance glycosynthase catalytic activity or product specificity.

### CROSS REFERENCE TO RELATED APPLICATIONS

This application claims priority under 35 U.S.C. § 119(e) to U.S. Provisional Patent Application No. 62/877,021, filed Jul. 22, 2019, which application is incorporated herein by reference in its entirety.

### STATEMENT REGARDING FEDERALLY SPONSORED RESEARCH OR DEVELOPMENT

This invention was made with government support under grant numbers 1904890 and 1704679 awarded by the National Science Foundation. The government has certain rights in the invention.

### SEQUENCE LISTING

The ASCII text file named “370602-7015US1(00056) Sequence,” created on Jul. 22, 2020, comprising 33.4 Kbytes, is hereby incorporated by reference in its entirety.

### BACKGROUND OF THE DISCLOSURE

Synthesis of glycan-based polymers (oligosaccharides and polysaccharides) using engineered carbohydrate-active enzymes (CAZymes) offers exquisite regioselective and stereoselective control over traditional synthetic chemistry approaches, which are atom inefficient and involve multi-step transformations. Glycosyltransferases (GTs) are naturally occurring CAZymes that synthesize glycans but give poor heterologous expression yields, have narrow substrate specificity, and use expensive nucleotide sugars, limiting the scale-up of in vitro glycans synthesis.

Chemoenzymatic synthesis using glycosyl hydrolases (GH) could permit production of complex glycans at high yields. GH are nature's antipodes of GT by hydrolyzing glycosidic linkages, but can also produce glycans via transglycosylation if the nucleophilic water is replaced by a sugar molecule as an acceptor. Unfortunately, transglycosylation suffers from low yields since the product is also a substrate for GH-mediated hydrolysis. However, most GH have plasticity in their structure, which allows for improving synthase activity.

Interestingly, glycosynthases (GSs) offer an alternative biosynthetic approach to producing glycans in a facile manner. The GSs are mutants of readily available microbial glycosyl hydrolases (GHs), which are incapable of hydrolyzing glycosidic bonds, and can be engineered to specifically synthesize complex glycans. However, to date, only a limited number of GSs have been created from wild-type GHs using an inefficient empirical strategy that have limited biosynthetic activity.

Unlike GTs, there is a much larger selection of GHs available that can be expressed readily in E. coli. Further, the active site GH nucleophile residue can be mutated to prevent product hydrolysis and improve product yields. However, the role of various accessory domains on the transglycosylation activity of mutant GH/GS is mostly unknown.

Thus, there is a need in the art for a method of identifying mutant GH/GS enzymes that allow for glycan production. The present disclosure fulfills this need.

### BRIEF SUMMARY OF THE DISCLOSURE

Disclosed herein is a method of determining if a protein has transglycosylase activity. In certain embodiments, the method comprises contacting the protein with an azido glycosyl donor and a glycosyl acceptor to form a system, and measuring any change in the azide concentration in the system. In certain embodiments, the azido glycosyl donor is substituted with an azido group at an anomeric carbon. In other embodiments, the azido glycosyl donor is substituted with an azido group at a non-anomeric carbon. In certain embodiments, the measurement of azide concentration comprises measurement of the concentration of an inorganic azide. In other embodiments, the measurement of azide concentration comprises the measurement of the concentration of an organic substituted azide, including azido glycosyl species.

In certain embodiments, the measuring step comprises contacting the system with a reagent comprising a strained alkyne coupling to a dye, under conditions that allow for reaction of the strained alkyne with any azide or azido compound present in the system. In certain embodiments, the reagent comprises bicycle[6.1.o]nonyne (BCN), dibenzocyclooctyne (DBCO), or any other strained alkyne. In certain embodiments, the reagent comprises 5-carboxytetramethylrhodamine (5-TAMRA), 6-carboxytetramethylrhodamine (6-TAMRA), or any combinations thereof. In certain embodiments, the strained alkyne and the dye are covalently linked by a linker in the reagent. In certain embodiments, the linker comprises a polyethylene glycol linker.

In certain embodiments, the measuring step uses a control protein that has no measurable transglycosylase activity or has a known transglycosylase activity. In other embodiments, the protein is a mutated glycosyl hydrolase (GH). In certain embodiments, the protein is expressed in a cell. In other embodiments, the cell comprises E. coli or P. pastoris. In certain embodiments, the system is within a cell (intracellular).

In certain embodiments, the measuring step comprises monitoring the fluorescence of the system. In other embodiments, fluorescence activated cell sorting (FACS) is used to separate individual cells by measured fluorescence. In other embodiments, the FACS is configured for high-throughput screening.

Disclosed herein are mutant polypeptide amino acid sequences of WT TmAfc-0306_(SEQ ID NO:1) comprising the mutation D224G (SEQ ID NO:2) and further comprising at least one additional mutation. In certain embodiments, the at least one additional mutation of the mutated construct (SEQ ID NO:2) is selected from the group consisting of L15K, N70D, A366V, T392S, K395N, D400A, T413P, I428T, and T429P. In other embodiments, the at least one additional mutation is selected from the group consisting of L15K-N7OD (SEQ ID NO:3); N70D-T392S (SEQ ID NO:4); N70D-T392S-A366V-K395N (SEQ ID NO:5); N70D-T392S-D400A (SEQ ID NO:6); N70D-T392-I428T (SEQ ID NO:7); and N70D-D400A-T413P-T429P (SEQ ID NO:8).

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1A: Two-step hydrolysis mechanism of WT Agrobacterium sp. β-glucosidase. First, the anomeric configuration of the sugar glycosidic bond (β linkage) is inverted to form the α-glycosyl-enzyme intermediate (GEI). The second step recovers the original anomeric configuration in the product (β-D-glucose). FIG. 1B: The E358A mutant glycosidase (or glycosynthase) cannot form a productive GEI. The enlarged binding site accommodates the α-glucosyl fluoride activated sugar donor.

FIG. 2A: One-step hydrolysis mechanism of an inverting glycoside hydrolase. FIG. 2B: One-step synthesis mechanism of an inverting GT employing a divalent cation. FIG. 2C: Two-step synthesis mechanism of a retaining GT, showing the GEI.

FIGS. 3A-3B: Two common human milk oligosaccharides (HMOs) used to test GH29 activity are (FIG. 3A) 2′-fucosyllactose (2′FL) and (FIG. 3B) 3′-fucosyllactose (3′FL).

FIG. 4A: The structure of the TmAfcA (PDB ID: 10DU), showing a bound fucose product, acid/base residue G266 (upper rendered side chain), and nucleophilic residue D224 (lower rendered side chain). FIG. 4B: Structure of glycosynthase TmAfcA D224G co-crystalized with fucosyl-azide, revealing that the overall protein structure is maintained in the mutant.

FIGS. 5A-5E: Computational methodology for uncovering CAZyme reaction mechanisms. FIG. 5A: If a solved crystal structure is unavailable, utilize homology modeling to predict CAZyme structure. FIG. 5B: Perform MD simulations to determine low energy conformations of reactant and product states. FIG. 5C: Informed by previously discovered transition state (TS) conformations, build multiple hypothesized TS structures. FIG. 5D: Using unbiased simulations, determine whether postulated TSs indeed lead to both reactant and product low-energy states. These are used to generate an ensemble of validated TSs to analyze for properties that correlate with reactivity, thus revealing the reaction coordinate. FIG. 5E: The resulting reaction coordinate allows determination of the energy required to overcome reaction barriers, which is converted to reaction rate coefficients that directly relate to experimentally observable activities.

FIG. 6A: Donor and acceptor sugars docked within CelE active site. FIG. 6B: Agar-CMC plate based GH-GS zone-clearing method. FIG. 6C: Some CelE mutants can efficiently synthesize oligosaccharides. FIG. 6D: SAXS suggests dynamic interaction of CBM with CelE catalytic domain drives TG activity.

FIG. 7: Phylogenetic tree of GH29 enzymes from published amino-acid sequences. The green arrows highlight the 3 enzymes to be studied to build the initial computational, predictive models. The enzymes highlighted in red are mutated, based on model predictions, to create new GSs. These genes were chosen to sample a diverse range of phylogenetically related sequences.

FIG. 8: Snapshots of the elementary synthesis step by TmAfcA D224G, joining 1-Azido-β-L-Fucose (1AF) to 4-Nitrophenyl β-D-xylopyranoside (4NX).

FIG. 9: Chemical rescue of TmAfcA mutants' hydrolytic activity on pNP-fucose using an external nucleophile confirms that the D224G mutant is a true GS.

FIG. 10A: HT-cloning and CAZymes synthesis using wheat germ cell free expression system. CAZymes directly assayed for (FIG. 10B) binding or (FIG. 10C) catalytic activity on glycans in a combinatorial manner.

FIG. 11A: Glycosynthase products docked in the active site of TmAfc-D224G based on quantum mechanical/molecular mechanics. FIG. 11B: Thin-layer chromatography (TLC) analysis of glycosynthase reaction of D224G with β-L-fucopyranosyl azide and pNP-β-D-xylopyranoside visualized with UV light. FIG. 11C: Reaction scheme of glycosynthase reaction of Tm0306-D224G with β-L-fucopyranosyl azide and pNP-β-D-xylopyranoside.

FIG. 12: SDS-PAGE protein gel for wild-type TmAFc0306_(WT), mutant D224A TmAfc0306_(A), mutant D224S TmAfc0306_(S), mutant D224G TmAfc0306_(G), and several epPCR derived purified mutant proteins identified by screening.

FIG. 13: TLC of glycosynthase reaction mixtures resulting from either wild-type (WT) or mutant D224G (Gly) with β-L-fucopyranosyl azide and pNP-β-D-xylopyranoside substrates.

FIG. 14: FACS based directed evolution approaches utilizing azide detection for screening glycan synthesizing CAZymes.

FIG. 15 illustrates the structure of fluorescent alkyne-DBCO-PEG4-FLUOR 545, as well as its reaction with sodium azide.

FIG. 16 illustrates absorbance analysis for the reaction of alkyne-DBCO-PEG4-TAMRA (or alkyne-DBCO-PEG4-Fluor 545) with sodium azide and glucosyl azide. UV spectra shows the loss of absorbance at 309 nm over time for both reactions.

FIG. 17 illustrates fluorescence analysis of click reaction of DBCO-PEG4-Fluor 545 and sodium azide and glucosyl azide in vitro. The alkyne to azide ratio is 1:2 in 1× PBS pH 7.4 buffer (37° C., 400 rpm, 5 hours).

FIGS. 18A-18B illustrates the fluorescence analysis of the SPAAC reaction between DBCO-PEG4-FLUOR 545 (200 μM) with sodium azide and β-D-glucopyranosyl azide in vitro at a temperature with a temperature of (FIG. 18A) 25° C. and (FIG. 18B) 10° C.

FIG. 19 provides the emission fluorescence as a function of excitation wavelength for SPAAC reaction products.

FIGS. 20A-20D illustrate the impact of free azide and triazole moieties on Rhodamine B fluorescence. FIG. 20A provides the structures of Rhodamine B and Fluor 545. FIG. 20B provides the relative fluorescence traces for mixtures of Rhodamine B with either organic or inorganic azide. FIG. 20C provides UV spectra for SPAAC reactions confirming the formation of triazole products from organic and inorganic azides with DBCO-NHS. FIG. 20D provides the fluorescence of a mixture of Rhodamine B with different triazoles independently as a function of time.

FIG. 21 provides a comparison of the fluorescence of the SPAAC reaction of glucosyl and fucosyl azide with DBCO-PEG4-Fluor 545 as a function of time.

FIG. 22 provides the fluorescence of SPAAC reactions of organic, inorganic, and an equimolar mixture of inorganic and organic azides with DBCO-PEG4-FLUOR 545.

FIG. 23A provides the protocol for sample preparation for confocal microscopy. FIG. 23B provides confocal microscopy images confirming permeation of DBCO-PEG4-FLUOR 545 dye into E. coli cells via Hoechst staining (0.1 μg/ml), bright field microscopy, and fluorescence with DBCO (0.1 mM).

FIG. 24 provides a proof-of-concept of the use of fluorescence assisted cell sorting (FACS) to identify and separate cells on the basis of the difference in fluorescence observed from triazole products formed in a SPAAC reaction in vivo.

FIGS. 25A-25B illustrates the in vivo click reaction of DBCO-PEG4-FLUOR 545 with sodium azide and β-L-fucopyranosyl azide, as monitored by FACS. FIG. 25A provides a scatter plot of the in vivo click reaction. FIG. 25B provides a fluorescence decay plot of the in vivo click reaction.

FIG. 26 provides flow cytometry characterization of fucosynthase (D224G) and wild type (WT) TmAfc enzyme expressing E. coli populations.

FIG. 27 provides FACS characterization of fucosynthase (D224G) and wild type (WT) TmAfc enzyme expressing E. coli populations.

FIG. 28 provides FACS based sorting of D224G mutant epPCR library with variation of the excitation wavelength used.

FIG. 29 illustrates the impact of free inorganic azide on initial E. coli growth rate.

FIG. 30 provides a conceptual overview of click-chemistry based ultrahigh-throughput screening (uHTS) for in vivo detection and sorting of improved glycosynthases.

FIG. 31A provides FACS scatter plots of the control and mutated libraries observed emplying an uHTS method. FIG. 31B provides a bar graph representing the chemical rescue activity for D224G cells and sorted mutants with improved GS activity. FIG. 31C provides the specific activity of the isolated M5 mutant (SEQ ID NO:6).

FIG. 32 provides a bar graph representing the chemical rescue activity for D224G cells and sorted mutants with improved GS activity.

FIG. 33 provides histograms showing the sampling along the reaction coordinate from each window in the umbrella sampling procedure described herein.

FIG. 34A provides the root mean square fluctuation of the M5 mutant fucosynthase structural model. FIG. 34B provides QM/MM simulations predicted free energy profile of the M5 mutant fucosynthase reaction.

FIG. 35A: Fucosylated glycans currently produced. FIG. 35B: Suite of complex fucosylated oligosaccharides.

FIGS. 36A-36C provides FACS scatter plots of an epPCR mutant library sorted to screen for mutants showing activity with β-L-fucopyranosyl azide as a donor sugar and common acceptor sugars associated with fucose: (FIG. 36A) galactose, (FIG. 36B) lactose, and (FIG. 36C) N-acetyl galactosamine.

### DETAILED DESCRIPTION OF THE DISCLOSURE

The present disclosure relates, in one aspect, to the discovery of a high throughput screening (HTS) method to rapidly screen for GH/GS variants that are generated using directed evolution techniques and that can significantly enhance glycosynthase catalytic activity or product specificity. A model fucosynthase from Thermotoga maritima has been developed for validation of this HTS method. Copper-free click chemistry reaction conditions were optimized for rapid quantification of azide-based products formed by active glycosynthase mutants using fluorescence. The difference in fluorescence profiles of wild type enzyme and mutants were analyzed using a flow cytometer. This click chemistry based screening technique was applied to the mutant library generated by random mutagenesis. In certain embodiments, this technique is a universal approach to screen for glycosynthases that have activated azide group on the donor substrate.

### Glycoside Hydrolases, Glycosyltransferases, and Glycosynthases

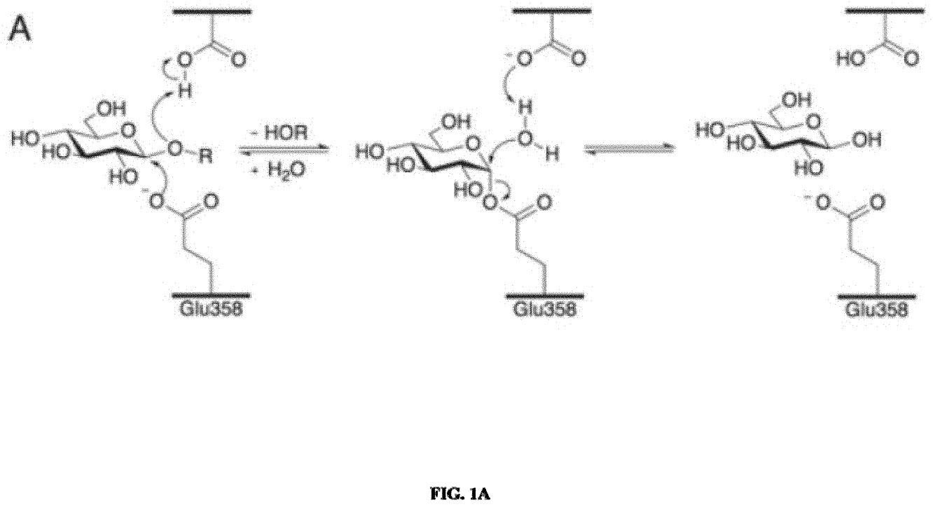

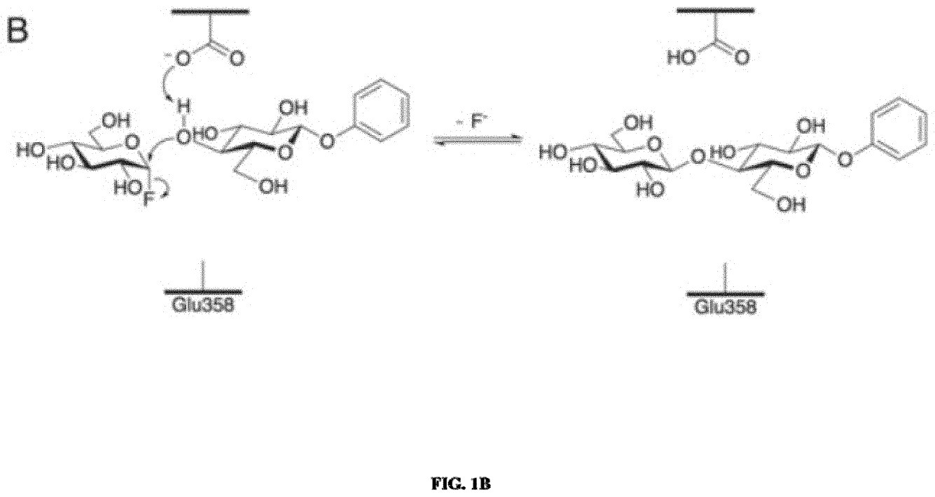

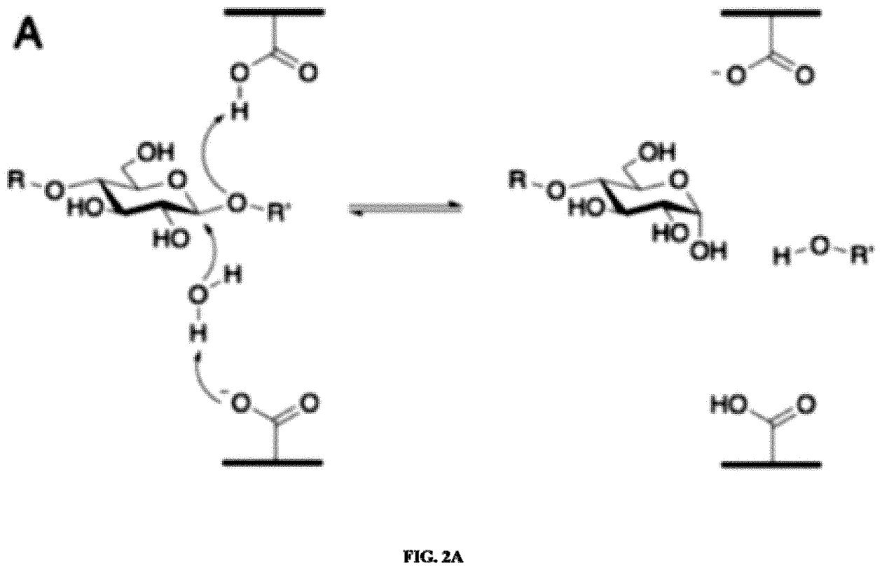

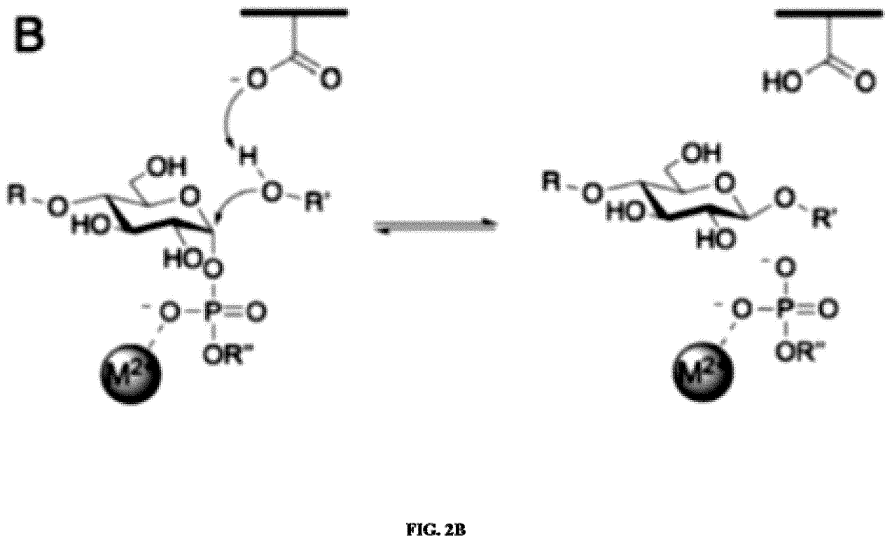

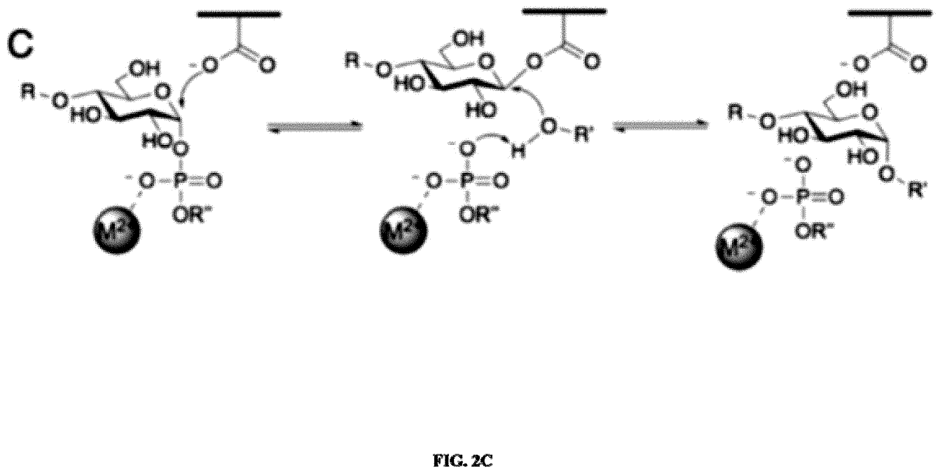

GHs and GTs are ubiquitous enzymes found in all kingdoms due to the central role of carbohydrates in life processes, as various oligosaccharide structures are used in diverse biological functions including signaling, energy storage, and structural components. GH enzymes cleave glycosidic bonds that join monomeric sugars to create oligosaccharides, and are grouped into families by amino-acid sequence similarity, now numbering 156 families as curated on the Carbohydrate-Active enZyme (CAZyme) database. Enzymes of each family are classified as either retaining or inverting, depending on whether the stereochemistry at the anomeric carbon is preserved between the reactant and product. Retaining enzymes (FIG. 1A) require a two-step hydrolysis mechanism, with a glycosyl-enzyme intermediate (GEI) characterized by a covalent bond between the cleaved substrate and the protein in the alternate orientation (in FIG. 1A, the GEI has an α-bond, as opposed to the β-orientation of the reactant and product). In inverting enzymes (FIG. 2A), a water molecule attacks the substrate while an acidic protein residue donates a proton to the glycosidic bond and a basic protein residue accepts a proton from the attacking water molecule. GH enzymes are often secreted extracellularly by organisms, and industrial processes routinely use them to break down carbohydrates, such as in converting plant biomass into simple sugars.

GTs fill an opposite function in nature, creating oligosaccharides by joining a sugar acceptor with an activated monomer, most commonly nucleotide diphosphate sugars such as UDF-glucose, UDP fructose, or GDP-mannose. in contrast to GHs, GTs operate mostly within cells (typically as membrane-associated proteins), and are less soluble and stable compared to GHs, and thus are less suited to industrial use. Further hampering their exploitation for production of oligosaccharides for research and industrial use is the high cost of generating sugar nucleotide substrates. In vivo synthesis can address some of these challenges by transferring or modifying the biosynthetic glycosylation pathways from desired eukaryotic or prokaryotic systems (e.g., Campylobacter jejuni) into genetically tractable and industrially relevant expression systems like E. coli or Pichia pastoris. However, glycosylation is an innately stochastic process leading to a complex milieu of glycoforms, making it challenging to produce a. defined library of glycans using such approaches alone.

Due to the difficulties of using GTs to create oligosaccharides, Gas have been explored for their potential to build glycosidic bonds, exploiting the innate ability of some GB enzymes to act as transglycosylases (TGs). The general mechanism is similar to the retaining mechanism shown in FIG. 1A, except that rather than nucleophilic attack by water, the attack is instigated by a hydroxyl oxygen on a sugar. Some enzymes have a higher natural propensity to act as TGs, and this activity can he enhanced by high sugar concentrations. However, the products are still potential reactants that can either be hydrolyzed or undergo further transgiycosylation, leading to low yields and a mixture of products, limiting their utility for bespoke oligosaccharides synthesis. The insight behind GSs is to enhance the ability of GHs to create glycosidic bonds and remove their capacity for glycosidic bond cleavage. Thus, GSs were created to perform the function of GTs without the need for expensive substrates. Derived from GHs, they retain the favorable GH characteristics of ease of expression and higher stability. Most GSs have been created from retaining GHs, although some have been created from inverting GHs. In those created from retaining GHs, the general approach has followed from the first engineered GS, illustrated in FIG. 1B. Specifically, the nucleophi lie residue is mutated to one that can no longer accept a proton, such as mutation from aspartate or glutamate to alanine, glycine, serine, and cysteine. In certain embodiments, the size of the side chains used in place of the WI nucleophile is an important factor in proper substrate positioning. In addition, the role of other residues within the active site or its vicinity on glycosynthase activity is even more poorly understood, GSs are characterized by the ability for catalytic rescue of hydrolytic activity in the presence of high concentrations (>1M) of an external nucleophile (e.g., azide or formate). To perform synthesis, GSs are supplied with sugars whose structures mimic the GEI. Like the native intermediates, these sugars will have the opposite stereochemistry at the anomeric center from the native reactant, and the anomeric carbon will be bonded to a leaving group such as fluoride or azide. These “donor” sugars are less expensive to produce than the activated, diphosphate sugars required by GTs. These donor sugars are paired with “acceptor” sugars that have high affinity for the binding site not occupied by the GEI mimic (termed the acceptor site in GSs), such as the phenyl-P-β-glucopyranoside shown in FIG. 1B. Since the native ability to hydrolyze glycosidic bonds is minimized for GS, there is no loss of product by subsequent cleavage. The requirement for the donor sugar to have an electrophilic leaving group, which is removed in the course of oligosaccharide synthesis, limits which species can serve as reactants, thus providing more control of the resulting product slate as compared to transglycosylation product distributions.

To date, only a limited number of GSs have been created, with between one and six members of any one family having been converted, and only from 17 GH families. The general empirical strategy has been to a) determine the nucleophilic catalytic residue, b) mutate that residue to alanine, glycine, serine, and/or cysteine, c) test for hydrolytic chemical rescue using external nucleophiles, and d) perform activity tests. This empirical approach is cumbersome and lacks the ability to screen growing genomic databases of CAZymes to identify the best targets using a theoretically based first-principles methodology.

### GH29 Enzymes

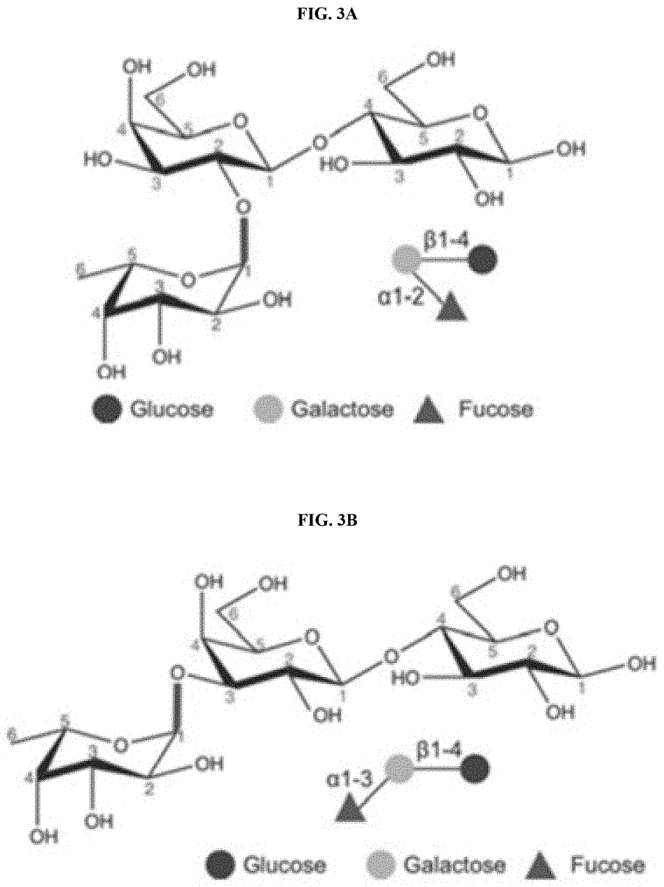

Of the 156 currently designated GH families, only two families contain α-fucosidases: family 29 (retaining hydrolases) and family 95 (inverting). As retaining enzymes have been more amenable to conversion into GSs, GH29 enzymes provide a promising route for creating enzymes to produce specific fucosylated oligosaccharides. The CAZyme database currently lists over 3,000 protein sequences classified as GH29 enzymes, with additional sequences continually deposited. The enzyme sources span archaea., bacteria, and eukaryota (from fungi to human). Of these, 33 have been characterized and show only α-fucosidase activity, breaking α-1,2-fucoside linkages (as in 2′-fucosyllacose, FIG. 3A) and/or α-1,3-fucoside linkages (as in 3′-fucosyllacose, FIG. 3B). The first structure to be solved was for TmAfcA in 2004, an α-1,2-fucoside (FIG. 4A). Since that time, structures have been solved for GH29 enzymes from seven additional organisms, and a structure of the GS TmAfcA D224G co-crystalized with α-L-Fuc-(1-2)-β-L-Fuc-N₃ in the donor site (FIG. 4B; Cobucci-Ponzano, et al, 2009, Chem. Biol. 16:1097-1108). The GSs TmAfcA D224G and SsFucAl D242S displayed varying activity with a β-L-Fuc-N₃ donor and a variety of acceptors, demonstrating that one GS can be used to create multiple oligosaccharides. Depending on the acceptor, between 1 and 5 different products were observed for a particular combination of donor-acceptor. In contrast, BfoAfcB D703 S produced only one reaction product for the five donor-acceptor pairs (same acceptors, but a β-L-Fuc-F donor) for which a synthesized compound was identified.

### Biological Role of Fucosylated Glycans and Current Synthesis Demand

The determination of finictional roles of glycans has been enabled by their commercial availability, but only a few such glycans are available, resulting in a limited understanding of glycans in living systems. Even so, it has become clear that fucosylated glycans play many key roles in biology, including mammalian use in ABO blood group antigens, host cells-gut microbe interactions, and selectin-dependent leukocyte adhesion. Also, non-digestible dietary glycans, together with mammalian gut host cells-produced glycans, represent critical energy sources that modulate the survival and proliferation of many microbial components of the gut microbiota.

Creating specific glycans by standard chemical synthesis is painstaking and expensive. Thus, biological routes are being pursued. As noted earlier, GSs have advantages for in vitro use, including lower cost compared to GTs and higher yield compared to GHs. Transglycosylation reactions using fucosidases has yielded inefficient routes (<5% yield) to synthesize fucosylated glycans. Much higher product yields of model fucosylated di-, tri-, and tetrasaccharides (30-50%) have been recently shown to be formed using β-fucosyl fluoride sugar donors and GS derived from both GH families 29 and 95. Due to the poor stability of β-glycosyl fluoride (vs. its α-anomer), there has been recent interest in exploring novel activated glycosyl donor sugars like β-fucosyl azides to produce glycans instead. Catalytic efficiency of GS employing non-native activated glycosyl donor substrates requires engineering of the active site residues, that cannot yet be predicted a priori using rational engineering approaches.

GHs are being rapidly discovered through cheaper sequencing of isolated microbial, microbiome, and metagenomic sources. These GHs offer a large library of enzymes that have not yet been exploited for engineering more effective and highly selective GSs. Directed evolution of GSs can be used to increase reaction rate and introduce novel substrate specificity. Additionally, isolated novel extremophilic GHs offer an opportunity to develop novel GSs with higher specific activity in non-aqueous solvents that would favor glycan (or glycoconjugate) synthesis and improve reactant and/or substrate solubility. However, one of the major challenges identified has been the lack of suitable high-throughput screening (HTS) methods for screening large GS libraries (>10⁶ mutants/day). A two-plasmid HTS method has been disclosed wherein one plasmid contains the GS gene while the other contains a screening enzyme that only releases a fluorophore from the product of the GS reaction but not the reactants (Bode, et al., 2016, Nutr. Rev. 74:635-644). Similarly, chemical complementation using a yeast three-hybrid system was used to link GS activity to the transcription of a reporter gene, making cell growth dependent on product formation (Lin, et al., 2004, J. Am. Chem. Soc. 126(46):15051-15059). Both of these approaches are highly specific to individual GS family and have narrow applicability to screen for novel substrate specificity. The first universal method to screen GS libraries (˜10⁴/day) using glycosyl fluoride as the sugar donor was a pH based assay (Ben-David, et al., 2008, Chem. Biol. 15(6):546-551). Here, hydrofluoric acid, a by-product of the GS reaction, was detected by a pH sensitive color indicator. A chemical probe that reacts specifically to the fluoride anion to generate a fluorophore has been used recently to screen small GS libraries (˜10²/day) (Andres, et al., 2014, Biochem. J. 458(2):355-363). However, to increase the probability of finding rarer GS mutants, screening techniques capable of handling much larger mutant libraries (10⁶-10³ mutants) are necessary. In one aspect, FACS based HTS methods alleviate the need to lyse cells, isolate plasmids, and retransform cells for iterative screening of much larger libraries. Directed evolution experiments for GSs are necessary to identify mutations both within and outside the active site region that can increase catalytic efficiency by >10²-10³ fold. The challenge is to use substrates without directly incorporating fluorophore tags to monitor GS reactions that typically bias substrate specificity.

### Computational Design of Engineered Enzymes

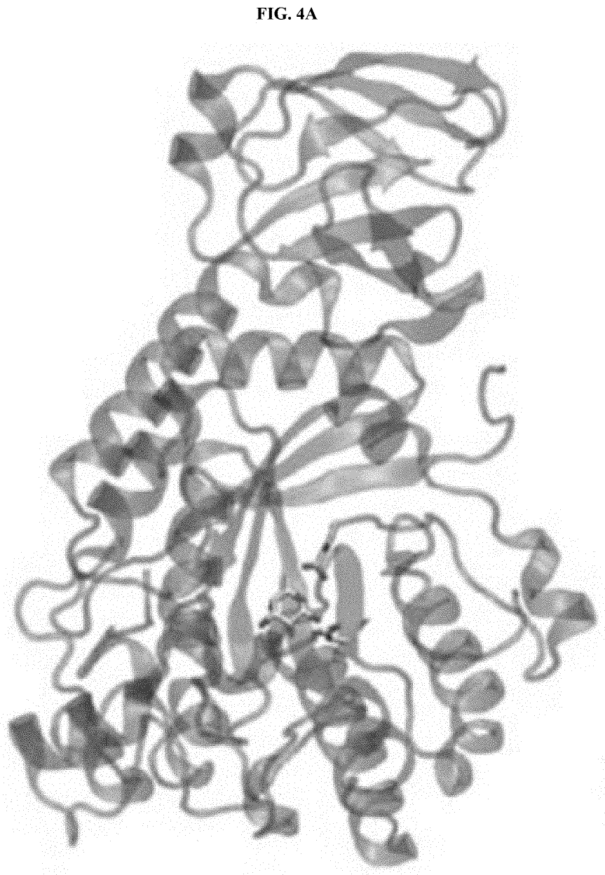

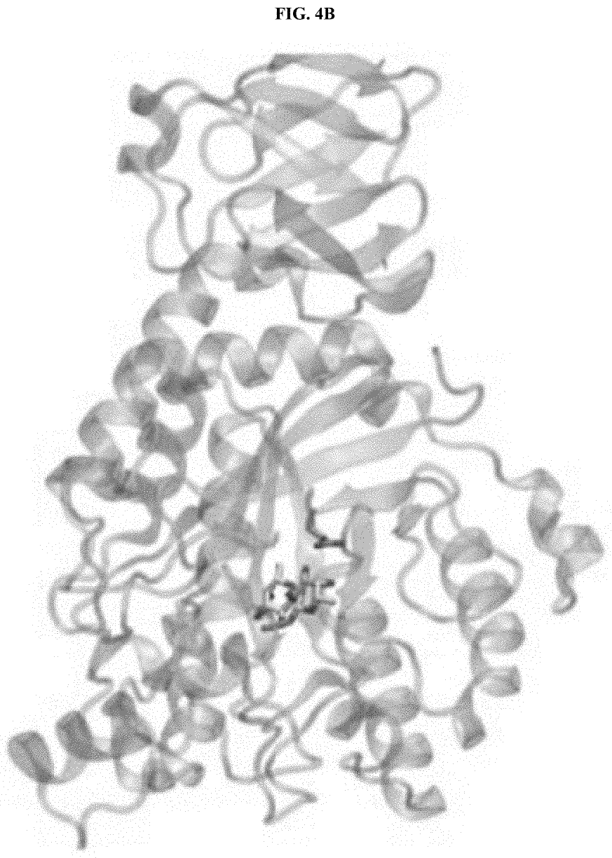

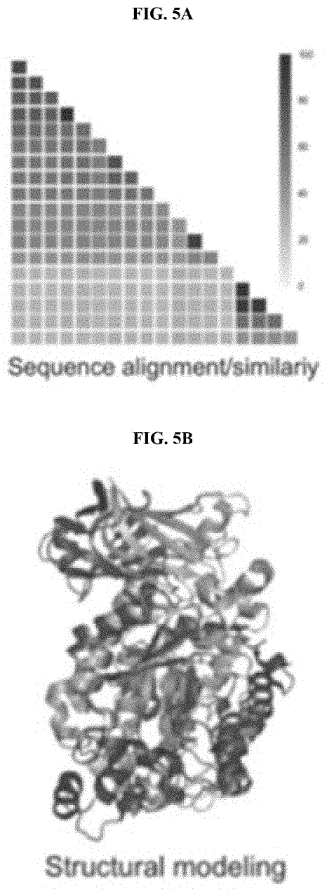

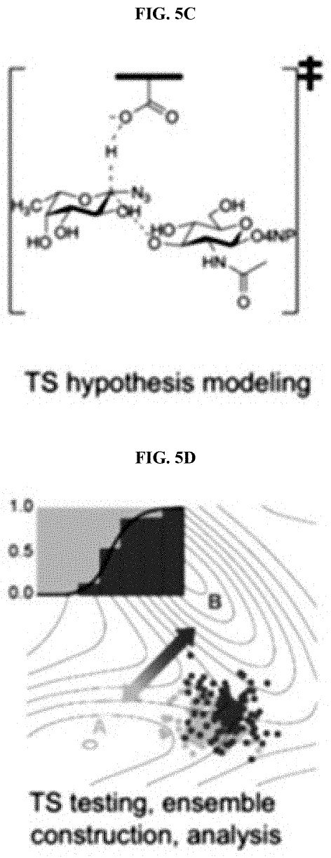

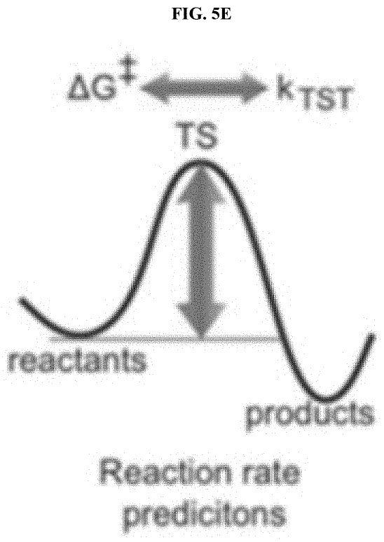

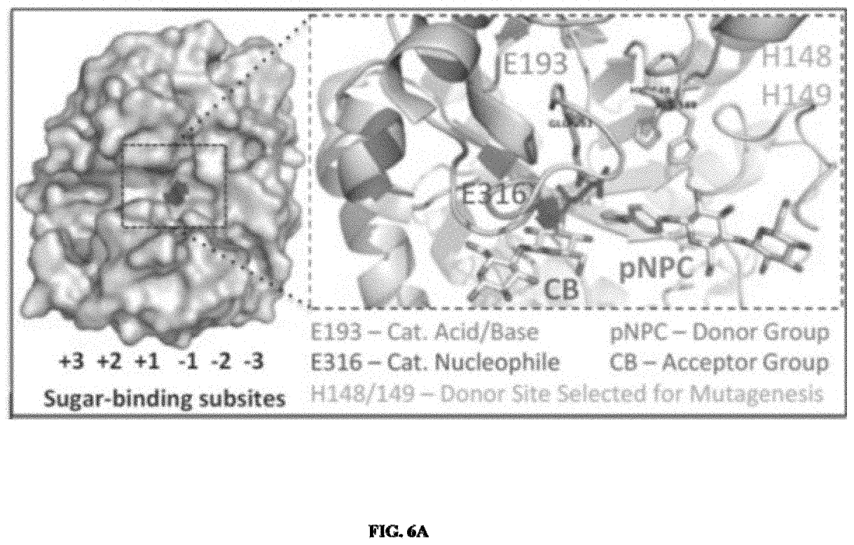

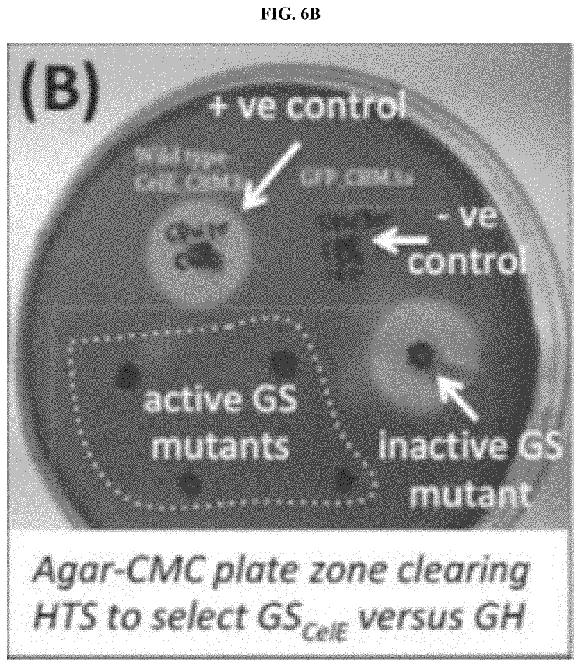

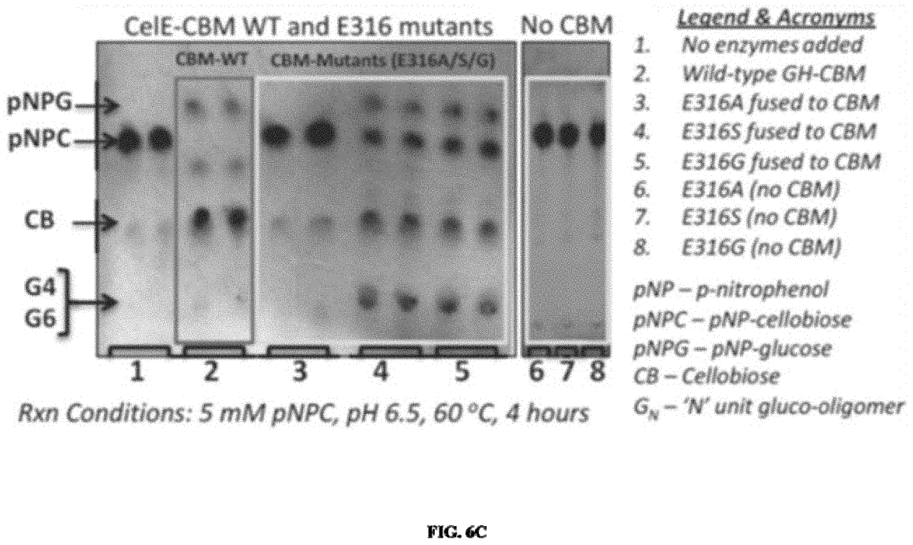

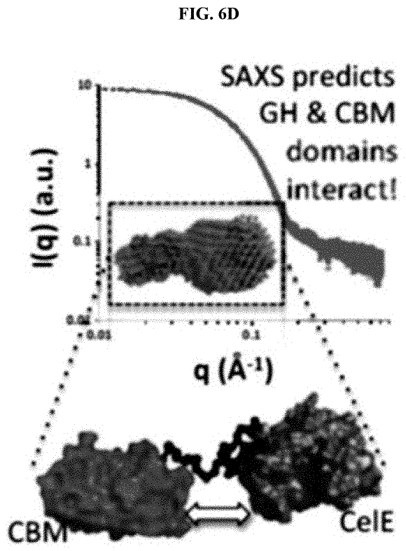

While computation is not required for design of novel enzyme, a semi-rational approach, combining computational insight into reaction mechanisms with experimental methods (such as directed evolution and mutational screening) can be used (FIG. 5A-5E). This combined approach can increase the success rate of experimental efforts. Computational approaches can harness the variety of experimental data available, from genomic sequences to three-dimensional structural characterization. The genomic data available has led to a variety of enzyme classification tools based on sequence similarity, which are harnessed in databases such as the CAZyme database to group enzymes into families that are likely to have similar activities, and building phylogenetic trees that can be used to postulate enzyme evolution and their implications for enzyme design. However, sequence information in isolation is generally insufficient to predict enzyme function. Structural information, usually from X-ray crystallography of the enzyme to be studied or built from a solved structure of a homologous enzyme, greatly aids in proper enzyme characterization, improving sequence alignment and providing clues as to which residues perform different functions. However, crystallization requires modifications of the environment and native substrates cannot be trapped in active conformations with WT enzymes. Instead, substrate mimics and/or inactive mutants can be co-crystalized to aid in active-site identification. Computational atomistic models can be generated using this experimental data, and the substrates and proteins modified to represent native molecules. These models of the native system can be used in molecular dynamics (MD) simulations to elucidate mechanisms and advance our understanding of structure-function relationships, using experimental activity studies to validate the models. Such simulations offer advantages over static models (such as Rosetta-based or docking models), as dynamic models can account for how the interactions between proteins and substrates lead enzymes to adapt to new, low energy conformations over the course of reaction, and thus provide more accurate predictions to direct targeted experiments for engineering more efficient enzymes. Additionally, the validated models can be used as a basis to test dynamic behavior of new mutants and/or non-native substrates can then be performed with the validated models.

In certain embodiments, a traditional Congo red dye assay for carboxymethyl cellulose (CMC) added to agar plates can be used for HTS of E. coil colonies that express active vs. inactive GHs. This allows one to identify protein mutants that have significant transglycosylation vs. hydrolytic activity on CMC based on ‘zone clearing’ about the colonies. However, this method cannot be used for screening GS capable of using activated sugar donors like β-glycosyl azide to synthesize non-glucosyl glycans.

Aspects of the present disclosure are described elsewhere herein.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, and molecular biology are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The following notation conventions are applied to the present disclosure for the sake of clarity. In any case, any teaching herein that does not follow this convention is still part of the present disclosure, and can be fully understood in view of the context in which the teaching is disclosed. Protein symbols are disclosed in non-italicized capital letters. As non-limiting example, “CelE” refer to the protein. Notations about mutations are shown as uppercase text. As non-limiting example, “E316G” refer to mutated site 316, where in a glutamic acid residue is replaced with a glycine residue.

As used herein the terms “alteration,” “defect,” “variation,” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

As used herein, the terms “conservative variation” or “conservative substitution” as used herein refers to the replacement of an amino acid residue by another biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, the terms “effective amount,” refer to a nontoxic but sufficient amount of an agent to provide the desired results. That result may be enhancing the rate of reaction, increasing purity of the product, increasing the yield of the product

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

“Instructional material,” as that term is used herein, includes a publication, a recording, a diagram, or any other medium of expression that can be used to communicate the usefulness of the nucleic acid, peptide, and/or compound of the disclosure in the kit for identifying or alleviating or treating the various diseases or disorders recited herein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

As used herein, the term “polypeptide” refers to a polymer composed of amino acid residues, related naturally occurring structural variants, and synthetic non-naturally occurring analogs thereof linked via peptide bonds.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

As used herein, the term “wild-type” refers to a gene or gene product isolated from a naturally occurring source. A wild-type gene is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

The disclosure provides a method of determining if a protein has transglycosylase activity.

In certain embodiments, the method comprises contacting the protein with an azido glycosyl donor and a glycosyl acceptor to form a system, and measuring any change in azide concentration in the system.

In certain embodiments, the azido glycosyl donor is substituted with an azido group at an anomeric carbon. In other embodiments, the azido glycosyl donor is substituted with an azido group at a non-anomeric carbon.

In certain embodiments, the measurement of azide concentration comprises measurement of the concentration of an inorganic azide. In other embodiments, the measurement of azide concentration comprises the measurement of the concentration of an organic substituted azide, including azido glycosyl species.

In certain embodiments, the measuring step comprises contacting the system with a reagent comprising a strained alkyne coupled to a dye, under conditions that allow for reaction of the strained alkyne with any azide or azido compound present in the system.

In certain embodiments, the reagent comprises bicyclo[6.1.0]nonyne (BCN), dibenzocyclooctyne (DBCO), or any other strained alkyne.

In certain embodiments, the reagent comprises 5-carboxytetramethylrhodamine (5-TAMRA), 6-carboxytetramethylrhodamine (6-TAMRA), or any combinations thereof.

In certain embodiments, the strained alkyne and the dye are covalently linked by a linker in the reagent.

In certain embodiments, the linker comprises a polyethylene glycol linker.

In certain embodiments, the measuring step uses as a control a protein that has no measurable transglycosylase activity or has a known transglycosylase activity.

In certain embodiments, the protein is a mutated glycosyl hydrolase (GH).

In certain embodiments, the protein is expressed in a cell.

In certain embodiments, the cell comprises E. coli or Pichia pastoris.

In certain embodiments, the system is within the cell (intracellular).

In certain embodiments, the measuring step comprise monitoring fluorescence of the system.

In certain embodiments, fluorescence activated cell sorting (FACS) is used to separate individual cells by measured fluorescence.

In certain embodiments, the method is configured for high-throughput screening.

In certain embodiments, disclosed herein, are mutant polypeptide amino acid sequences of WT TmAfc-0306_(SEQ ID NO:1) comprising the mutation D224G (SEQ ID NO:2) and further comprising at least one additional mutation.

In certain embodiments, the at least one additional mutation of the mutated construct

(SEQ ID NO:2) is selected from the group consisting of L15K, N70D, A366V, T392S, K395N, D400A, T413P, I428T, and T429P.

In other embodiments, the at least one additional mutation of D224G (SEQ ID NO: 2) is selected from the group consisting of L15K-N7OD (SEQ ID NO:3); N70D-T392S (SEQ ID NO:4); N70D-T392S-A366V-K395N (SEQ ID NO:5); N70D-T392S-D400A (SEQ ID NO:6); N70D-T3925-1428T (SEQ ID NO:7); and N70D-D400A-T413P-T429P (SEQ ID NO:8).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and assaying conditions with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, practice the claimed methods of the present disclosure. The following working examples therefore, specifically point out the preferred embodiments of the present disclosure, and are not to be construed as limiting in any way the remainder of the disclosure.

### Methods

### Gene Synthesis and Cloning

A model GH family 29 fucosidase enzyme, referred to as Tm-alpha-fucosidase (TmAfc) from a hyperthermophile Thermotoga maritima was selected for modification. The native (or wild type) gene Tm0306_that encodes TmAfc was optimized for E. coli expression and custom synthesized with AsiSI and BamH1 restriction sites specific flanking residues in pUC57 by Genscript Biotech Corporation (Piscataway, N.J.). The Tm0306_gene was sub-cloned from Genscript's pUC57 vector into our customized pEC vector (with T5 promotor & Kanamycin selection marker) using standard restriction cloning. The catalytic nucleophile of Tm0306_ (D224) was independently mutated into alanine (D224A), serine (D224S), and glycine (D224G) using standard site-directed mutagenesis protocols. For mutagenesis, 0.5 μM of forward and reverse primers for mutagenesis were mixed with 20 ng of plasmid DNA in a 10 μl reaction volume. The reaction was carried out using 1× Master Mix (Phusion DNA polymerase, 200 μM dNTPs, 1× Phusion HF buffer, 1.5 mM MgCl₂) with 5% DMSO and the reaction volume was made up to 10 μl by adding nuclease free PCR water. Amplification was confirmed by gel electrophoresis before the PCR amplified reaction mixtures were digested with 10 U of Dpn1 enzyme (New England Biolabs) at 37° C. for 1 hour. The Dpn1 digested mixture was transformed into E. Cloni 10 g competent cells (Lucigen, Wis.) using the Zymo transformation kit and plated onto LB agar plates with appropriate selection marker (Kanamycin). Several random colonies were selected, plasmid DNA was extracted, and verified by DNA sequencing (Genscript, Piscataway, N.J.).

| | | Tₘ |
|---|---|---|
| Primer name | Primer sequence | (° C.) |
| Tm0306_D224A_Forward | (SEQ ID NO: 9) | 69 |
| | GATGTTCTGTGGAACGCC | |
| | ATGGGTTGGCCGGAG | |
| Tm0306_D224A_Reverse | (SEQ ID NO: 10) | 69 |
| | CTCCGGCCAACCCATGGC | |
| | GTTCCACAGAACATC | |
| Tm0306_D224S_Forward | (SEQ ID NO: 11) | 67.3 |
| | GATGTTCTGTGGAACTCC | |
| | ATGGGTTGGCCGGAG | |
| Tm0306_D224S_Reverse | (SEQ ID NO: 12) | 67.3 |
| | CTCCGGCCAACCCATGGA | |
| | GTTCCACAGAACATC | |
| Tm0306_D224G_Forward | (SEQ ID NO: 13) | 69 |
| | GATGTTCTGTGGAACGGC | |
| | ATGGGTTGGCCGGAG | |
| Tm0306_D224G_Reverse | (SEQ ID NO: 14) | 69 |
| | CTCCGGCCAACCCATGCC | |
| | GTTCCACAGAACATC | |

### Protein Expression and Purification

Sequence verified wild type (Tm0306_WT) and corresponding nucleophile mutant

(Tm0306_D224A/S/G) DNA plasmids were transformed into E. coli BL21 (DE3) competent cells and plated onto LB agar plates with 50 μg/ml kanamycin. Individual colonies were picked to inoculate a 50 ml starter culture of LB media supplemented with kanamycin antibiotic (50 μg/ml) and incubated at 37° C. for 12-16 hours. Overnight grown cultures were transferred into 1000 ml LB media containing 50 μg/ml kanamycin and grown at 37° C. until the culture density reached an OD₆₀₀ of 0.4-0.8. The protein expression was then induced using 0.5 mM Isopropyl β-D-1-thiogalactopyranoside (IPTG) and cultures were incubated at 25° C. for 20 hours. The cell pellets were recovered by centrifugation and stored in freezer until needed. The cell pellets were suspended in lysis buffer (20 mM sodium phosphate, 500 mM NaCl and 20% glycerol, pH: 7.4) in a 1:5 ratio of cells to buffer solution (total weight basis), along with protease inhibitor cocktail (1 μM E-64, 0.5 mM benzamidine and 1 mM EDTA) and lysozyme (10 μg/m1) and lysed by sonication on ice. The lysed pellets were then centrifuged and the cell lysate supernatant enriched in the desired soluble protein was recovered. The N-terminal his-tagged proteins of interest were separated from the other undesired E. coli proteins using an IMAC (Ni-immobilized metal affinity chromatography) column using the NGC-FPLC system (Bio Rad, Hercules, Calif.). Briefly, the Ni-IMAC column was equilibrated with the IMAC binding buffer (100 mM MOPS, 10 mM imidazole, 500 mM NaC1, pH: 7.4). Next, the cell lysate supernatant was loaded onto the column and the IMAC binding buffer was run through the column to remove any non-specifically bound proteins from the column. The protein of interest was next eluted with the IMAC elution buffer (100 mM MOPS, 500 mM imidazole, 500 mM NaC1, pH 7.4). The protein was buffer exchanged using desalting columns (GE Healthcare, Catalog number: 17-0851-01) into 10 mM of 2-morpholin-4-ylethanesulfonic acid or IVIES at pH 6. The purified protein concentration was estimated using the Spectradrop UV spectrophotometer (SpectraMax M5e) based on 280 nm absorbance. Purity of all enzymes was confirmed by SDS-PAGE based on gel densitometric analysis using pre-cast stain-free (Bio-Rad) protein electrophoresis gels.

### Fucosidase Activity and Chemical Rescue Assays

The activity of the purified enzymes Tm0306_WT, Tm0306_D224A, Tm0306_D224S, and Tm0306_D224G was evaluated using pNP-F (4-nitrophenol α-fucopyranoside) as substrate procured from Carbosynth Limited. In each experiment, 1 μg of protein was added to 2 mM pNP-F added in a reaction buffer containing 50 mM MES pH 6 and incubated at 60° C. for 1.5 hours. Blank wells with pNP-F alone were taken as buffer/substrate but without the added proteins as controls. Three replicates were taken for each reaction mixture. After 1.5 hours of the reaction, 100 μL of the reaction mixture was transferred to a transparent 96-well microplate along with 100 μL of 1 M NaOH and the absorbance was measured at 410 nm using a UV/Vis spectrophotometer (SpectraMax M5e) to determine total released pNP absorbance upon substrate hydrolysis. A pNP calibration curve was built to find the relationship between the measured absorbance and estimated concentration. In order to recover or ‘rescue’ the hydrolytic activity of the hydrolytically inactive nucleophile mutants, high concentrations of external nucleophiles like sodium azide and sodium formate (2 M each) were additionally added to reaction mixtures and incubated at 60° C. for 2 hours. After the reaction was completed, 30 μL of the reaction mixture was transferred to a transparent 96-well microplate and mixed with 70 μL of DI water and 100 μL of 0.1 M NaOH. The absorbance was measured at 410 nm using a UV/Vis spectrophotometer (SpectraMax M5e).

### Glycosynthase In Vitro Activity Assays

For evaluating the glycosynthase activity of Tm0306_WT and Tm0306_D224G, 40 μg of the protein was added to a mixture of 10 mM β-L-fucopyranosyl azide (Catalog number: 66347-26-0, Chemily Glycosciences) and 50 mM pNP-0-D-Xylose (Carbosynth Limited) and incubated at 60° C. for 24 hours in 50 mM IVIES buffer pH 6.0. Two replicates were taken for each reaction mixture. The reaction mixture was then analyzed using Thin Layer Chromatography (TLC) using Silica Gel 60 F254 TLC plates from Merck. The mobile phase used for TLC was ethyl acetate: methanol: water (at 70:20:10 v/v ratios). Standards were also run on the TLC plate to determine the unknown detected spots in reaction sample based on retention factor (R_{f}) value. The plate was epi-illuminated and directly imaged under UV light at wavelength λ=305 nm to visualize pNP and pNP-containing compounds. The plates were then sprayed with visualization solution containing 0.1% orcinol dye in 10% H₂SO₄, then dried and heated at 100° C. for 15 min to visualize reducing sugars and acid-labile sugars.

### In Vitro Strain Promoted Azide-Alkyne Cycloaddition (SPAAC) Reaction

DBCO-PEG4-Fluor, a commercially available click-chemistry reagent comprising a red fluorophore dye and a dibenzyocyclooctyne moiety connected by a PEGylated linker, was reacted with either sodium azide (inorganic azide) or β-D-glucopyranosyl azide (organic azide) at 37° C. in 1× pH 7.4 PBS buffer for a total reaction time of 5 hours. The strain-promoted azide-alkyne cycloaddition (SPAAC) reaction kinetics were monitored continuously during the 5 hour period.

### Exogenous Free Azide Rhodamine-B Fluorophore Studies

200 μM Rhodamine-B was mixed with 400 μM of azide (independently sodium azide and β-D-glucopyransoyl azide) in 1× PBS buffer pH=7.4. Separately, 200 μM Rhodamine-B in 1× PBS buffer pH=7.4 without an azide was taken as the Rhodamine-B only control. An azide only control was prepared with 400 μM of an azide independently in 1× PBS buffer pH=7.4 without Rhodamine-B. Each reaction was incubated at 37° C. for 3 hours and the fluorescence spectra for the each respective solution was recorded every 30 minutes at 550 nm excitation, 570 nm auto cutoff and 590 nm emission in a UV spectrophotometer SpectraMax M5e. Respective azides were mixed with Rhodamine-B and the mixture fluorescence was recorded at various time points at 550 nm excitation, 570 nm auto cutoff and 590 nm emission using UV spectrophotometer Spectra Max M5e.

### Exogenous Free Triazole Rhodamine-B Fluorophore Studies

200 μM of DBCO-NHS was mixed with 400 μM azides (sodium azide and β-D-glucopyransoyl azide) in 1× PBS buffer pH=7.4 to allow the SPAAC reaction to take place. Here, 200 μM of DBCO-NHS with 1× PBS buffer pH=7.4 without azides was taken as the DBCO-NHS control. Azides were taken with 1× PBS buffer pH=7.4 without DBCO-NHS as azide controls. Only 1× PBS buffer pH=7.4 was taken as the blank for the reaction. The reaction was incubated at 37° C. for 200 min at 400 rpm. The SPAAC reaction was monitored by absorbance at 309 nm at varioius timepoints. After 200 mins total reaction time, 200 μM of Rhodamine-B dye was added to all wells, including controls, and incubated at 37° C. while constantly measuring fluorescence at 550 nm excitation, 570 nm auto cutoff and 590 nm emission using a spectrophotometer SpectraMax M5e for various incubation times ranging from 0-120 minutes from the point of addition of the dye.

Glucosyl- and Fucosyl-Azide SPAAC Reaction with DBCO-PEG4-Fluor 545

The SPAAC reaction was performed at 37° C. using DBCO-PEG4-Fluor 545 with either glucosyl azide or fucosyl azide with a reaction time of approximately 320 minutes, while constantly measuring fluorescence at 550 nm excitation, 570 nm auto cutoff and 590 nm emission using a spectrophotometer SpectraMax M5e.

### In Vitro Quantitative Detection of Inorganic and Organic Azides

The SPAAC reaction was performed with each of 100% sodium azide, 100% β-D-glucopyransoyl azide, and 50% sodium azide/50% β-D-glucopyransoyl azide, independently under typical SPAAC conditions with DBCO-PEG4-FLUOR 545 and the fluorescence at 550 nm excitation, 570 nm auto cutoff and 590 nm emission was monitored using a spectrophotometer SpectraMax M5e.

### Confocal Fluorescence Microscopy

Starter culture was inoculated with E. coli BL-21 (DE3) glycerol stock for pEC_Tm0306_WT plasmid in 10 ml LB media with 50 μg/ml kanamycin. Here, 5 ml LB media with 50 μg/ml kanamycin alone was taken as a control. The starter culture and the control were incubated at 37° C. for 16 hours. Next, 2.25 ml of the starter culture was transferred to 45 ml minimal media with 45 μl kanamycin and 5 ml minimal media with 5 μl kanamycin was taken in a separate tube as control and incubated at 37° C. for 16 hours until OD600 of Tm0306_WT reached about 2. The cell culture was centrifuged at 8,000 rpm for 15 minutes and the supernatant was discarded. The culture was washed thrice with equal amount of 1× PBS buffer pH 7.4 and centrifuged at the same conditions as described above. The washed culture was now re-suspended in same amount of 1× PBS buffer pH 7.4. OD600 was measured again and it was found to be 2 again which remains in consistency with the amount of cells in the culture before the washing step. First, 2 tubes (labeled as C1 and C3) were prepared as control for the experiment with 200 μl cells and 200 μl DI water. Another 2 tubes (labeled as C5 and S1) were prepared with 200 μl cells and 66 μl of 0.5 mM of DBCO-PEG4-FLUOR 545. All tubes (C1, C3, C5 and S1) were incubated at 37° C. for 30 minutes. The samples were centrifuged at 10,000 rpm for 3 minutes and the supernatants were discarded. Now, 50 μl of freshly prepared 4% paraformaldehyde was added to all the samples, mixed well, and incubated at 37° C. for 10 minutes. The samples were centrifuged at the same conditions as described above and the supernatants were discarded. The cell pellets obtained were washed twice with 1× PBS buffer pH=7.4 followed by re-suspending in 266 μl of 1× PBS buffer pH=7.4, mixing well, and centrifuging at 10,000 rpm for 3 minutes and finally discarding the supernatant. Next, 50 μl of 0.1 μg/ml Hoechst 33342 was added to S1, mixed well and incubated at 37° C. for 10 minutes. S1 was centrifuged at 10,000 rpm for 3 minutes and the supernatant was discarded. S1 was washed twice with 1× PBS buffer pH=7.4 and the supernatants were discarded. C1, C3, C5 and S1 were re-suspended in 100 μl of 1× PBS buffer pH=7.4 and mixed well. Next, 2 μl of the samples were mixed with 50 μl mounting media (Prolong diamond antifade mounting agent, Catalog number: P36965, Thermo Fisher Scientific) in PCR tubes and centrifuged to remove bubbles. Finally, 10 μl of the samples were placed on a glass slide covered with transparent glass cover slip and incubated at 25° C. for 24 hours in dark and visualized under a confocal microscope.

### In Vivo SPAAC Reaction and Flow Cytometry

Click chemistry reaction between DBCO-PEG4-Fluor545 and Azide (NaN3 or Glc-N3) was performed in-vitro at 1:2 ratio at 37° C. for 4 hours. The click chemistry reaction mixture was next incubated with 500 μl of E. coli cells at OD=1 for 1 hour. Samples were then run using a flow cytometer (Beckman Coulter CytoFLEX Cytometer) to characterize single-cell fluorescence and the overall cell population distribution. Blue laser was used for excitation (488 nm) and red fluorescence channel filter was set at 585/42 nm BP. Here, data from two independent flow cytometry runs per sample (biological replicates) were used for subsequent analysis. A total of 10,000 events per sample run were captured using flow cytometer and the median in vivo fluorescence observed for all replicate sample runs is reported below. For gating, control cells incubated with sodium azide and glucosyl azide alone were taken as control cells and the fluorescence obtained from the cells is excluded.

### Fluorescence Activated Cell Sorting (FACS) Flow Cytometry

Flow cytometry (Guava EasyCyte) was done using 488 nm excitation and 583 nm emission filters, while FACS (MoFlo Cell Sorter) was done using 488 nm excitation and 575 nm emission filters. This experimental data provided a proof of concept in-vivo validation for difference in signals obtained for an active glycosynthase vs. an inactive enzyme control (WT) using both a flow cytometer and FACS instruments. Although the difference in signal was marginal due to the poor activity of D224G.

Error-Prone PCR (epPCR) via Sequence Ligation Independent Cloning (SLIC)

For insert PCR, 0.5 μM of forward and reverse primers were mixed with 20 ng of plasmid DNA of Tm0306_WT with 0.2 mM of dATP and dGTP, 1 mM of dCTP and dTTP in a 100 μl total reaction volume. The reaction was performed in 1× Taq buffer with 1.25 U of Taq DNA polymerase. 0.1 mM and 0.5 mM MnCl₂ was taken in different tubes with (labeled as I1 and 12) and without (labeled as 13 and 14) 1.5 mM and 7 mM MgC12. For Vector PCR products, 0.5 μM of forward and reverse primers were mixed with 20 ng of plasmid DNA of Tm0306_WT in 1× Phusion Master mix in a 50 μl total reaction volume (labeled as V1 and V2).

| | | Tₘ |
|---|---|---|
| Primer name | Primer sequence | (° C.) |
| Tm0306_WT_epPCR_Vector_Forward | (SEQ ID NO: 15) | 57.5 |
| | GAATAAGGATCCTCT | |
| | AGAGTCGAC | |
| Tm0306_WT_epPCR_Vector_Reverse | (SEQ ID NO: 16) | 57.7 |
| | CATGGCGATCGCCT | |
| | GG | |
| Tm0306_WT_epPCR_Insert_Forward | (SEQ ID NO: 17) | 57.7 |
| | CCAGGCGATCGCCA | |
| | TG | |
| Tm0306_WT_epPCR_Insert_Reverse | (SEQ ID NO: 18) | 57.5 |
| | GTCGACTCTAGAGG | |
| | ATCCTTATTC | |

### PCR Conditions Used for Insert PCR Product Amplification:

| Process | Temperature (° C.) | Time (s) |
|---|---|---|
| Initial Denaturation | 95 | 60 |
| Denaturation | 95 | 30 |
| Annealing | 60 | 30 |
| Extension | 68 | 180 |
| Final Extension | 68 | 300 |
| Hold | 10 | — |

No. of cycles (20)

For Vector PCR products, 0.5 μM of forward and reverse primers were mixed with 20 ng of plasmid DNA of Tm0306_WT in 1× Phusion Master mix in a 500 total reaction volume (labeled as V1 and V2). PCR conditions used for Vector PCR:

| Process | Temperature (° C.) | Time (s) |
|---|---|---|
| Initial Denaturation | 98 | 30 |
| Denaturation | 98 | 10 |
| Annealing | 60 | 30 |
| Extension | 72 | 180 |
| Final Extension | 72 | 300 |
| Hold | 10 | — |

No. of cycles (30)

### DNA Gel for PCR Amplification Check, PCR Product Purification

Once PCR is complete, 2 μl of the PCR product was mixed with 3 μl PCR water and 1 μl of the Purple loading dye and run in SYBR safe DNA gel alongside 5 μl of DNA ladder at 120 V for 40 minutes. With the remaining PCR products, PCR product purification was performed using PCR extraction kit from IBI Scientific.

### Dpn1 Digestion, SLIC and Transformation

Reaction mixtures were prepared for Dpn1 digestion. Next, 100 ng of V1 was taken without insert as a control (Reaction-1), 100 ng of V1 was taken with Il in the Vector: Insert ratios of 1:2.5, 1:5 and 1:10 (Reactions 2,3 and 4 respectively), 100 ng of V1 was taken with 12 in the Vector: Insert ratios of 1:2.5, 1:5 and 1:10 (Reactions 5,6 and 7 respectively), 100 ng of V1 was taken with 14 in the Vector: Insert ratios of 1:2.5, 1:5 and 1:10 (Reactions 8,9 and 10 respectively) in 1× Cut smart buffer in a 10 μl total reaction volume and were digested using 20U of Dpn1 at 37° C. for 1 hour. After DPnl digestion, 1.5U of T4 DNA Polymerase in NEB buffer 2.1 was added to the PCR reaction mixture in a total reaction volume of 20 μl and incubated at 25° C. for 5 minutes for SLIC (Sequence Ligation Independent Cloning). The PCR products were incubated on ice immediately after the SLIC run and transformed into E.cloni 10 g cells and incubated at 37° C. for 2 hours. The transformation mixture was plated on LB-agar plate with 50 μg/ml kanamycin and incubated at 37° C. for 16 hours. Several colonies were observed on the LB agar plates and colony screening was performed to figure out the right colonies.

### Colony Screening

For colony screening, 30 random colonies were picked from Insert plate (Reaction 3), 30 random colonies were picked from Insert plate (Reaction 9), 5 random colonies were picked from Vector plate (Reaction 1) and transferred to a PCR plate (PCR plate-1) with 5 μl PCR water and incubated at 95° C. for 5 minutes. Also, the tip which was used to pick up a particular colony was transferred to LB media with 50 μg/ml kanamycin and incubated at 37° C. for 14-15 hours. 1 μl of colony from the PCR plate 1 was added to 0.5 μM Ncol forward (TTGCTTTGTGAGCGGATAAC) and 0.5 μM T7 terminator reverse (GCTAGTTATTGCTCAGCGG) primers. The reaction was performed in ix Master mix in total reaction volume of 40 μl in PCR Plate-2. After colony screening PCR was complete, 2 μl of the PCR reaction mixture was added to 3 μl PCR water and 1 μl of the Purple loading dye alongside 5 μl of the DNA Ladder and loaded onto a DNA gel and run at 120 V for 40 minutes. The DNA gel was imaged using Gel Doc EZ Imager and the positive colonies were identified. The positive colonies were purified using PCR extraction kit and sent for DNA sequencing. The grown colonies were also sent for DNA sequencing after performing mini-prep plasmid extraction for epPCR mutation rate analysis.

FACS Sorting of epPCR Library

The error-prone PCR was generated and validated as described in the error-prone PCR (epPCR) via sequence ligation independent cloning (SLIC), Dpn1 digestion, SLIC and transformation, and colony screening sections. The epPCR mixture was run on a DNA gel and the bands were extracted using gel extraction. The epPCR products were purified using the PCR clean-up kit from IBI Scientific. Dpn1 digestion was performed at 37° C. for 1 hour and SLIC was performed at 25° C. for 5 minutes on the extracted products. The SLIC reaction mixture was transformed into E.cloni 10 g cells and incubated at 37° C. for 2 hours in SOC media for recovery. After 2 hours, the transformation mixtures were directly transferred to 5 ml LB media as inoculum and grown at 37° C. for 16 hours. Next, 1 ml starter cultures were transferred to 20 ml volume cultures in conical flasks with suitable antibiotics and incubated at 37° C. for around 2-3 hours until OD₆₀₀ reached the exponential phase (OD600=0.4-0.8). Then, 1 mM IPTG was added to the cultures and incubated at 37° C. for 1 hour to induce protein expression. OD600 was measured after one hour of IPTG induction and 1 ml of the cell cultures were taken out into a sterile micro-centrifuge tubes and centrifuged twice and the supernatants in each round were discarded. Cells were washed twice with 1× PBS buffer pH=7.4 and then re-suspended in 60 μl of 1× PBS pH 7.4 with 10 mM β-L-Fucosyl azide and 25 mM pNP-Xylose added to makeup a total reaction volume of 150 μl. This solution was then incubated at 37° C. for 2 hours for the glycosynthase reaction to take place. After 2 hours, the samples were centrifuged and supernatants were discarded. The samples were then re-suspended in PBS buffer and 50 μM DBCO-PEG4-Fluor 545 was added into the total reaction volume of 150 μl and incubated at 37° C. for 30 minutes. After 30 minutes, the samples were centrifuged to remove supernatant. Unstained cell samples and D224G (i.e., template DNA) were also taken as controls. The samples were then re-suspended in 1 ml of 1× PBS buffer pH=7.4, filtered using 40 μm filter and run on a FACS instrument (BD Influx High Speed Sorter) with 561 nm excitation laser.

### HPLC Analysis of GS Reaction Products

GS reactions were performed for D224G and the FACS M5 purified proteins to evaluate their specific activities. Briefly, 300 pmoles of each purified protein was reacted with 1 μmole of β-L-fucopyranosyl azide and 25 μmoles of pNP-β-D-Xylose in a 100 μl reaction volume at 60° C. Distinct reaction mixtures were setup for sampling different GS reaction timepoints (i.e., 2 h, 6 h, 10 h, 16 h, 24 h) and three reaction replicates were used for each time point. After each time point, the tubes were rapidly frozen at −20° C. to quench the reaction and stored for HPLC-UV analysis. The HPLC analysis was performed on a Shimadzu HPLC system. Briefly, a mobile phase of 90:10 (Acetonitrile:Water) was run through a HILIC column (Shodex Asahipak NH2P-50; 4E 4.6×250mm) until a stable baseline is achieved prior to sample injection. Next, 5 μl reaction mixture was injected onto the column and all pNP-based products (i.e., pNP-xylose, α-L-Fuc-(1,4)-β-D-Xyl-pNP, and αL-Fuc-(1,3)-β-D-Xyl-pNP) were detected using a DAD detector at 254 nm and 300 nm absorbance wavelengths. The raw data was acquired and analyzed using Shimadzu Lab Solutions software. Three distinct peaks were obtained for substrate pNP-Xylose and both GS products for which their respective peak areas were calculated. The area for pNP-Xylose peaks in blank samples was used to normalize and estimate the concentrations of each product in the reaction samples. The initial product formation rate was calculated using the data for 5% conversion of substrate and normalized with the amount of protein added to determine the specific activity of each protein. A two-sided Students t-test was performed for the specific activities of D224G and FACS M5 protein to compared and evaluate their statistical significance.

### Molecular Modeling and Simulations

The molecular model used here was based on a previously published model for the D224G single mutant of the same enzyme. Molecular mechanics (MM) simulations were performed using the Amber 18 software suite. A transition state structure from the previous study was mutated further to match the M5 construct, minimized over 2500 steps, heated from 100 to 300 K over 30,000 2-fs steps, and finally equilibrated over 5 ns with a restraint in place to keep the substrates in the previously identified transition state. The simulations used an Andersen thermostat with a randomization period of 100 steps, a cutoff distance of 8 Å, and the SHAKE algorithm to restrain bonds with hydrogen atoms.

To prepare the system for umbrella sampling, beginning from the equilibrated MM structure the system was further equilibrated over 100 1-fs steps using combined quantum mechanics/molecular mechanics (QM/MM) simulations with the same QM region from the original study, without restraints. Within the QM region the same 8 Å cutoff was used, but SHAKE was not. Because there were no restraints, the system naturally relaxed into one energetic basin (reactants in this case). From there, gentle restraints with initial weight zero and increasing by 0.025 kcal/mol-Å ² each step were used to guide the substrates to the other basin, and this simulation was run until the substrates reached the defined product state. Then, the trajectory was divided into evenly spaced windows along the reaction coordinate every 0.5 units from −11 to 9 (the reaction coordinate is unitless), with the initial coordinates for that window taken from the frame of the trajectory closest to the window center. Using the rxncore model implement in a modified version of Amber, five independent umbrella sampling simulations were performed on these windows, each with step size 0.5 fs and harmonic restraint weight 20 kcal/mol, were run in each window for between 1,811 and 5,437 steps (average 3670.2) each, of which first 1,500 steps were discarded for equilibration. The free energy profile was constructed using pymbar version 3.0.5. The samples were decorrelated using the pymbar.timeseries.subsampleCorrelatedData function to ensure only independent samples were considered.

The M5 construct model was also used to perform five unbiased 10-ns MM simulations (of which the first 2.5 ns of each was discarded for equilibration) and compared to the same number and length of simulations for the single (D224G) mutant system. The average by-residue root-mean-square fluctuations (RMSF) were calculated using pytraj and subtracted from one another to produce the ΔRMSF data.

### Example 1: Identification of Certain Structural Features that Determine whether a Particular GH29 Mmutant will become a GS

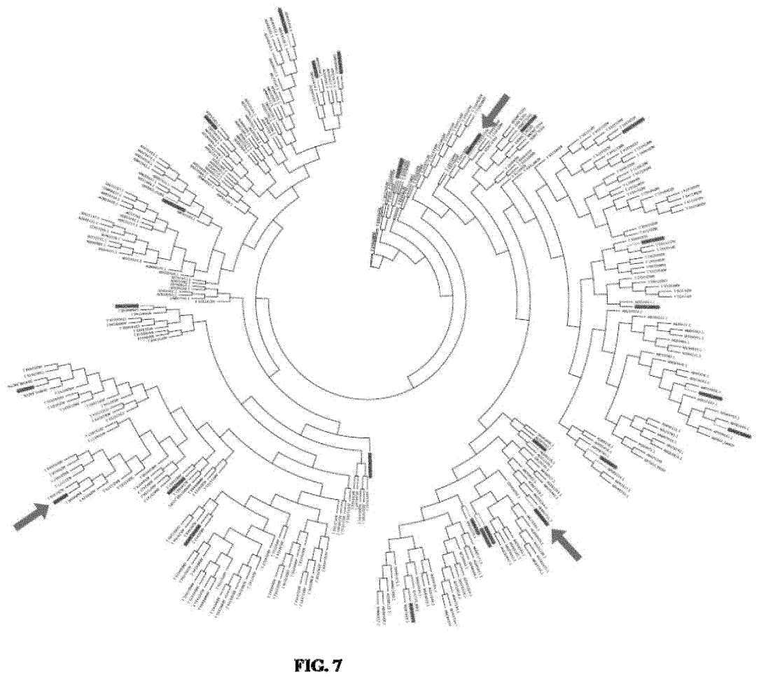

Building on efforts to create GSs from three GE129 enzymes representing a diversity of sequences from this family, as indicated by their distance on a phylogenetic tree (FIG. 7), one can determine which portions of the enzyme are responsible for their differing behavior by studying the series of mutations that produce an active or inactive GS for each enzyme. Three GH29 enzymes have been converted to GS enzymes by mutating the aspartate nucleophile to a smaller, uncharged residue: TinAfcA :D224G, SsFucAl D242S, BbAfcB D703G, and BbAfcB D703 S were all active. One can study the α-L-fucosidases (and their mutants) from B. longum subsp. infantis instead of B. bifidum; these enzymes are highly homologous (96% sequence identify), and BlAfcB has a solved crystal structure. The TmAfcA D224G mutant acts as a GS.

Effort on determining mechanistic studies on GS enzymes will include building atomistic models for all three enzymes (starting from crystal structures or homologs). As part of this effort, one can also create a Python-based module to streamline making homology models for CAZymes as a first step toward the development of in silico tools to screen such enzymes with knowledge of their amino acid sequence alone. One can leverage existing sequence alignment algorithms (e.g. Multi Seq), and refine the alignment based on conserved motifs of GH29 enzymes, including identifying and aligning the nucleophilic and acid/base residues, and SWISS-MODEL for homology modeling. This model can be developed and tested while making homology models for BbAfcB from the closely related BiAfcB enzyme structure and SsFucA1 from Fusarium graminearum Fco1.

Using atomistic models, low-energy conformations of each of the three GS enzymes in complex with reactants (β-fucosyl-azide and 4NP-β-D-GlcNAc) or products (α-1,3-filcosyl-4NP-β-D-GlcNAc) can be determined using replica-exchange MD. The substrates were chosen based on experiniental studies that show high (86%) reaction efficiency to one product. Postulated transition-state (TS) conformations are created informed by these simulations and solved Michaelis complex structures. They are used as the basis of transition path sampling simulations of the synthesis reaction. The advantage of this approach over other types of enhanced sampling methods such as metadynamics is that a reaction path does not have to be selected a priori, and no bias is added to the forces propagating the dynamics. With this method, one generates ensembles of thousands of trial TS geometries, tested with short simulations to determine if they can serve as intermediates in a reactive trajectory (connecting the reactants and products). The resulting data on which geometries lead to reactive trajectories are interrogated to determine the physical properties (such distances and angles between atoms) that correlate with reactivity, as the PI has shown previously for a GH6 enzyme. Such simulations can reveal unintuitive parameters that are vital for reaction, as the key parameters determining reactivity were those describing the nucleophilic water molecule orientation, which is extremely difficult to determine through wet-lab experiments. This approach sheds light on whether there is an optimal size for the active site cavity that can be quantified and used to predict what side chain should be substituted for the native nucleophile to induce GS activity. In certain embodiments, this work allows for the development of models that can be adapted to other GH29 mutant enzymes, allowing predictions of how to convert yet-unstudied GHs into GSs.

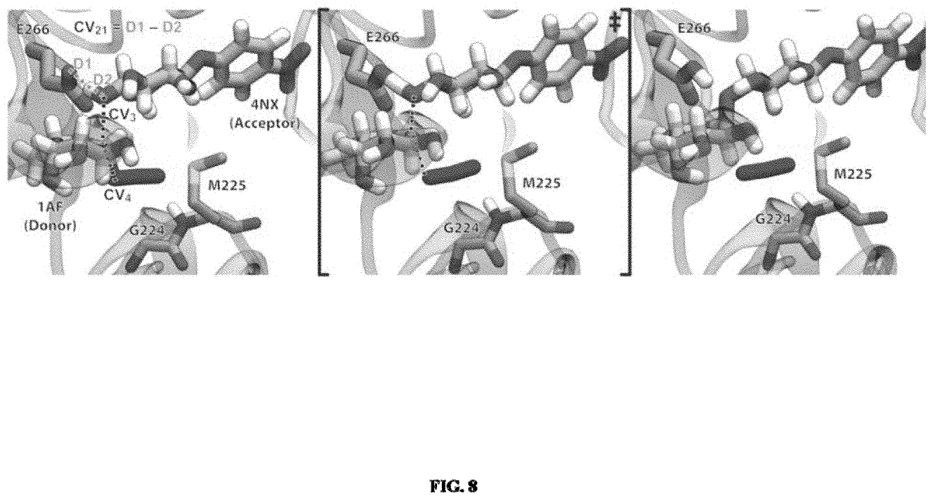

The first step in mechanism-based rational design of GH29 glycosynthases is to understand the mechanism for at least one such enzyme. The simulation of TmAfcA found a single reaction barrier for the synthesis step and it was endothermic. However, the barrier was ˜7 kcallmol (leading to a rate coefficient 8 orders of magnitude higher), and the enzyme-bound product was only 3 kcallmol higher than the enzyme-bound reactant, with an overall exothermic reaction by 1.3 kcal/mol. Significantly, the methodology introduces no bias into the simulations, and one is able to mine the simulations to determine which features (e.g., residue properties) are key to the reaction, and which can be modified to improve reaction efficiency. This extraction of structure-function relationships forms the basis of how one determines which mutations to make in the active and binding sites, For example, in TmAfcA D224G, functional requirements for activity were identified in the lack of residues that stabilize departure of the leaving group, and limited space to accommodate it in the active site, explaining why larger side chains in that position (e.g., serine instead of glycine) lead to inactive glycosynthases. This may also provide insight to potential structural changes to fill that need: Met-225, shown in FIG. 8, is positioned such that a mutation to a polarizable or positively charged side chain could lead to a more active enzyme. One can advance our structure-function correlation algorithms to identify promising sites for multiple GS mutations and use Rosetta to investigate longer-timescale phenomena, such as checking that multi-site mutants do not impede protein folding.

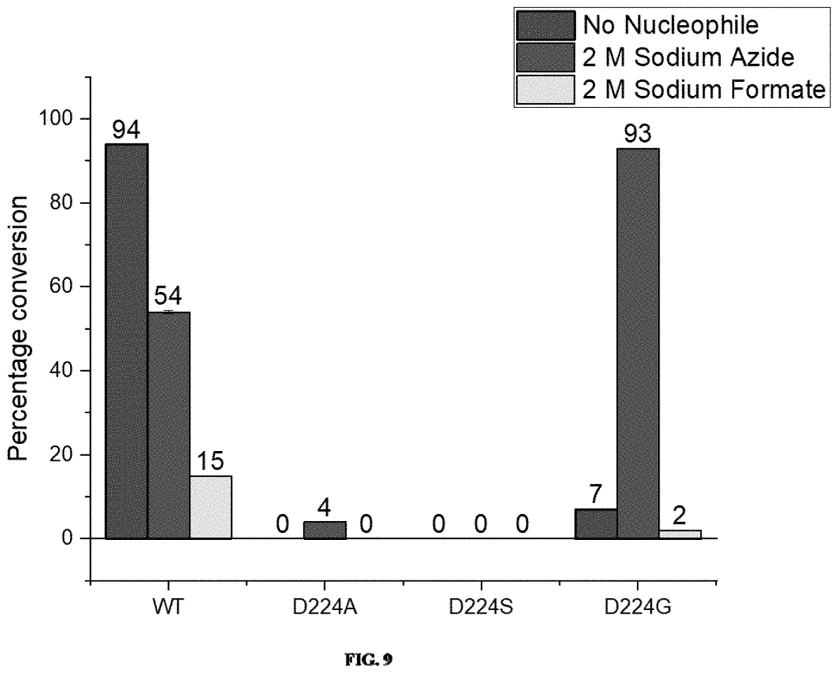

Model predictions have been tested (FIG. 9). GH 29 genes and their respective nucleophile mutants are generated first, followed by other genes representative of the entire tree to synthesize fucosylated oligosaccharides using for example β-fucosyl fluoridelazi de as donor sugar and lactose as the acceptor sugar. In certain non-limiting embodiments, residues around. the donor sugar binding sites can impact the catalytic efficiency of the GS by assisting in the departure of the azide leaving group. Therefore, engineered GS libraries with single/multiple mutations at substrate or product binding sub-sites are designed computationally and produced in vitro.

### Example 2: Prediction and Testing of which Mutants of Disparate GH29 Enzymes are Active Fucosynthases

Using an automated, streamlined process for homology modeling of α-L-fucosidases and their mutants, one can computationally test which mutations change the active site analogously to the previously successful mutations to fucosynthases. These mutants are then synthesized and tested for activity, allowing model refinement, if needed.

Phylogenetically related GH 29 genes (˜25-30 total) identified from genomic sequences (FIG. 7) are engineered into GSs. DNA libraries (150-200 total mutants) are designed in silico, based on the Rosetta scores, for all single mutations at the nucleophilic site (e.g., to Alanine, Glycine, Serine, Cysteine, or Asparagine). Libraries are generated using overlap extension polymerase chain reaction (PCR) with degenerate oligonucleotide primers (IDTDNA) and gBlocks gene fragments Gibson assembly. Type-B and Type-C family CBMs that are known to bind galactose/lactose (e.g., CBM 13 and CBM 32) and other HMO-sugar monomers are also fused to target GS library to further identify novel CBM-GS interaction constructs that might give higher HMO yields. A protein library of over 12 families of Type-A and Type-B native CBMs from diverse microbial sources that specifically bind to glycans is available. GS constructs are sub-cloned into the custom cell-based pEC (T5 promoter) and cell-free pEU vectors as N-terminal His tagged constructs. Single colonies are screened by colony PCR methods and DNA sequencing by Genewiz (Piscataway, N.J.).

Cell-free protein expression is used for preliminary HTS of GS activity using desired donor and acceptor sugars (FIG. 10B). Flexi-vector cloning facilitates easy transfer of gene libraries between the pEU-pEC plasmids. Cell free synthesis can be directly coupled with CAZyme activity screening without purification of synthesized proteins (FIG. 10C). Using customized reagents from Cell Free Sciences™, even disulfide containing CAZymes (e.g., CBM1) can be produced in a functional form.

GS mutants are expressed in a 96-well high-throughput format and activity determined. The cell free system is compatible for detection of reducing sugars (DNS colorimetric assay), p-nitrophenol (UV absorbance), or click-chemistry compatible products (fluorescence) with high sensitivity and without interference from the wheat germ background. GSs can give significant variation in product yields by changing reaction conditions. Therefore, reactions are carried out in 384-well microplates for each mutant to screen the following conditions; enzyme loading (0.5-5 pM), donorlacceptor loadings (1-50 mM), pH (pH 5,5-8,5), temperatures (45-65° C.), and reaction times (1-24 hours). Donor sugars with alternative leaving groups (e.g. p-nitrophenol, fluoride or azide) for optimizing donor sugar addition to diverse acceptor groups can be used. Product formation is monitored by in-situ detection of leaving group released using a microplate reader and TLC analysis to confirm oligosaccharides formation. Our multi-tiered HTS approach allows one to identify highly active GS mutants for subsequent detailed characterization of GS activity.

Mutant G-S selected are expressed on :large-scale using BL21 strains (about 50-250 ml), IMAC purification, and desalted into a low molarity MOPS buffered saline for detailed activity characterization. Both BL21 and Rosettagami strains can be used to obtain correctly folded, fully functional CelE and other CAZymes. if needed, mutants can be expressed periplasmically. Typical expression yields for GSceiE is about 150 mg/L, therefore one can readily generate all mutants. One can utilize HPLC and LC-MS/MS methods for glycan characterization. Detailed structural characterization of products can be done using NMR and/or MALDI-TOF-MS/MS. To explore non-nucleophilic site mutations, a larger library of GS mutants can be generated using error-prone PCR or other targeted mutagenesis techniques for screening using fluorescence-activated cell sorting (FACS) based methods.

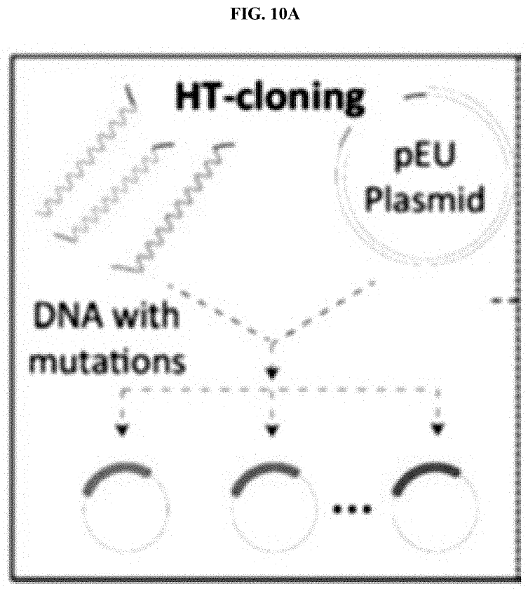

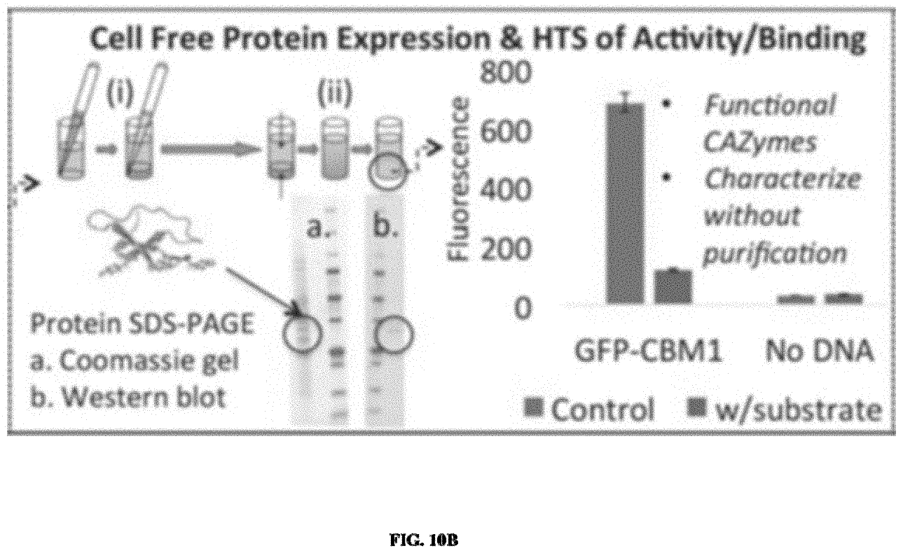

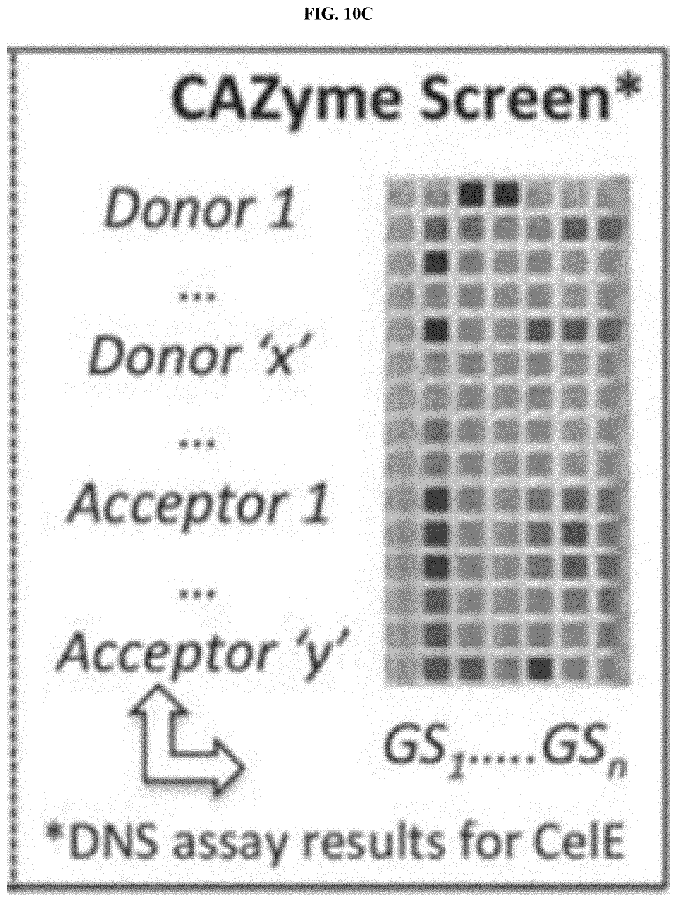

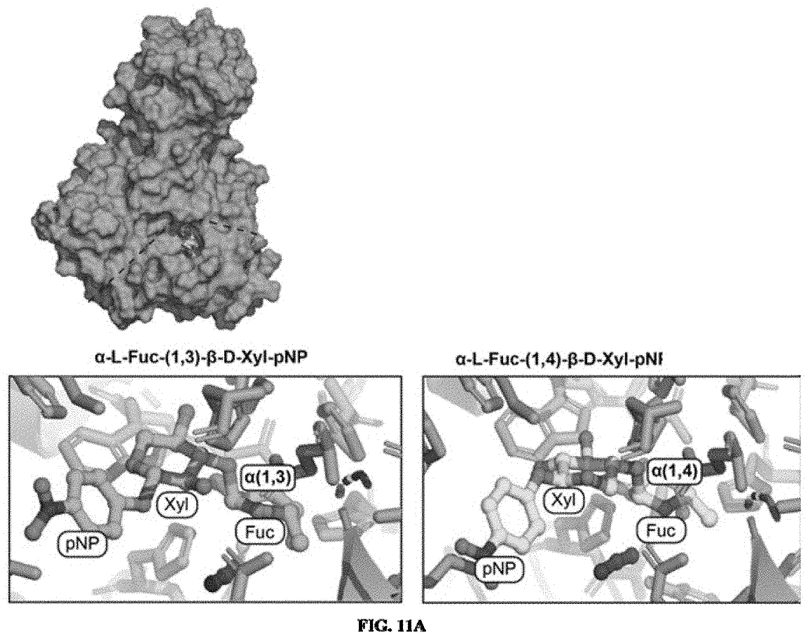

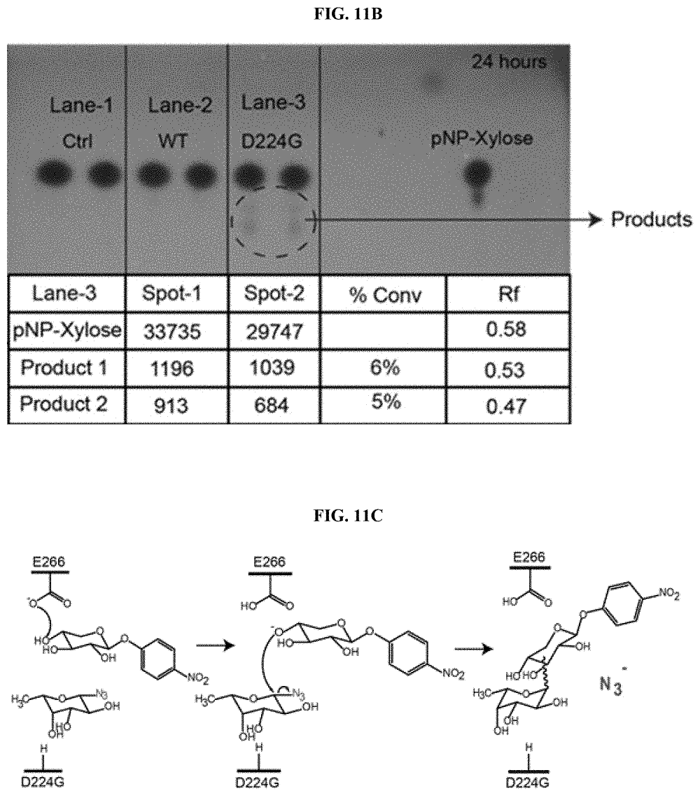

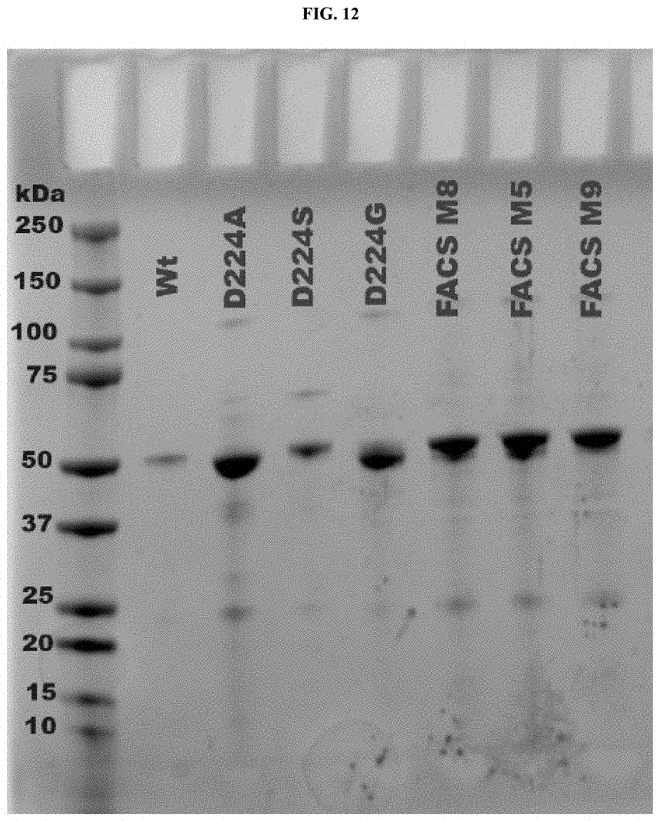

In certain embodiments, an α-L-fucosidase enzyme (Tm0306 gene) isolated from Thermotoga maritima, was selected as a model GS enzyme for mutagenesis, bacterial expression, and further in-vitro testing (FIG. 11A). This fucosidase (TmAfc0306) has been engineered to an α-L-fucosynthase by mutating the catalytic nucleophile D224 residue to each of alanine, glycine, and serine independently, and the mutant fucosynthases D224A, D224G, and D224S respectively, were purified by SDS-PAGE (FIG. 12).

Chemical rescue experiments on the mutant fucosynthases demonstrated that an exogenous azide nucleophile was sufficient to rescue the hydrolytic activity of the glycine mutant (D224G) by 98% while the alanine mutant (D224A) and the serine mutant (D224S) did not show any significant recovery in activity (FIG. 9).

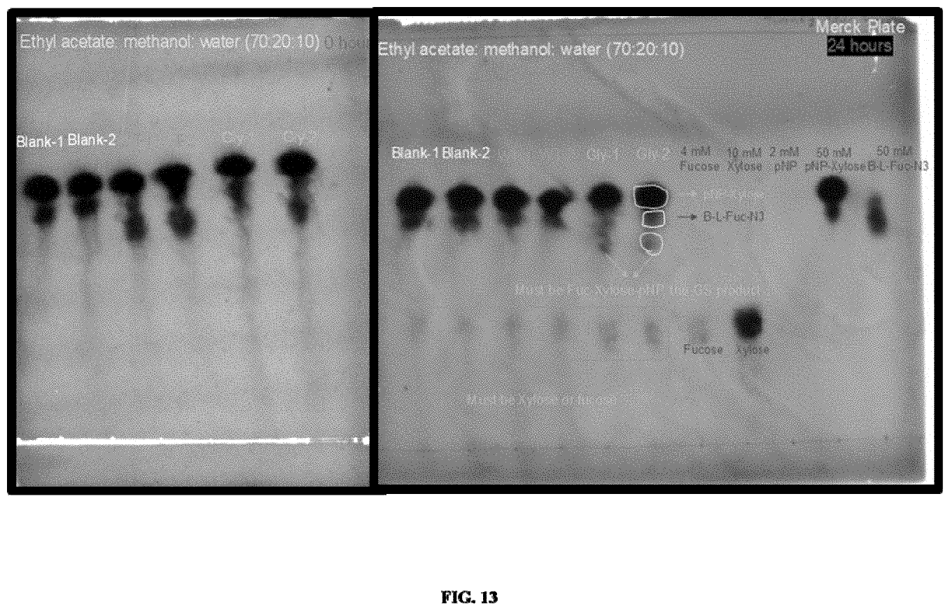

The in vitro reaction of pNP-β-D-xylose (acceptor sugar) and β-L-fucosyl azide (donor sugar) with purified D224G mutant resulted in the formation of only two minor glycosynthase products α-L-Fuc-(1,4)-β-D-Xyl-pNP (55%; molar basis) and α-L-Fuc-(1,3)-β-D-Xyl-pNP (45%; molar basis) (FIG. 11B and FIG. 13).

Without wishing to be bound by theory, a mechanism for the fucosynthase reaction has been proposed (FIG. 11C). The reaction is initiated by an acid/base reaction between a residue of the D224G and the —OH group at the C4 position of pNP-β-D-xylopyranoside, affording a deprotonated pNP-β-D-xylopyranoside anion. This alkoxide proceeds to attack the anomeric carbon of β-L-fucopyranosyl azide present adjacent to the catalytic nucleophile D224G site facilitating the release of the azide leaving group. Finally, pNP-β-D-xylopyranoside would form a glycosidic bond with β-L-fucosyl moiety to produce β-L-fucopyranoside-β-D xylopyranoside-pNP as the final GS reaction product.

While the reaction was successful, the total GS reaction product yield was found to be only about 6% (i.e., based on initial pNP-β-D-xylose starting concentration), even after a prolonged reaction incubation period of several days, indicating that the D224G GS activity is very low. Thus, the D224G construct was used as the baseline GS for the development of an assay method to identify additional mutants in a high throughput manner.

### Example 3: Development of an In Vivo Detection Method for GS Activity

In another embodiment, one can use a novel click-chemistry method for detection of glycosyl azides as sugar reactants (or released azide products) for in-vivo detection of GS activity. This method allows one to screen a large library of variants for targeted GS genes by using fluorescence activated cell sorting (FACS). FACS methods have been used to identify mutations for GTs and GHs (but not GSs yet) that increase catalytic efficiency by >10²-10³ fold. There are currently no HTS methods available to facilitate directed evolution of GSs capable of using activated sugar donors like β-glycosyl azide to synthesize glycans. Unlike pNP, fluoride and azide are smaller in size and are more likely to be tolerated within the active site. However, the major drawbacks with existing fluoride detection based HTS methods for GS are: i) low sensitivity limit (0.01-10 mM range) for detection of reaction products that reduces throughput and makes it challenging to fine-tune selection threshold, ii) the inability to distinguish between desired GS activity oligosaccharide products versus side-reaction products due to self condensation of donor sugars or hydrolysis of glycosyl fluorides due to poor stability in aqueous conditions (e.g half-life ranges between 0.25-10 days for most α- and β-anomers), and iii) the lack of a fluorophore than can directly detect unreacted glycosyl fluoride. In certain embodiments, one advantage of using glycosyl azides as substrates for GS reactions is that the azide moiety can be selectively conjugated to fluorophores using Staudinger click chemistry under conditions compatible with in vivo reaction conditions. Glycosyl azides can also be readily chemically synthesized using one-pot reactions from unprotected sugar monomers as well as produced enzymatically at high yields unlike glycosyl fluorides. In certain embodiments, this disclosure provides a universal glycosyl azide based HTS assay that can be used for directed evolution of GSs and applied to develop highly efficient chemoenzymatic routes for designer fucosylated glycans synthesis.

The present studies include the development of a HTS methodology for detection of glycosyl azide (and/or azide anion) as a marker of GS activity and the sorting of intact E. coli cells using FACS to screen a large library of GH 29 variants.

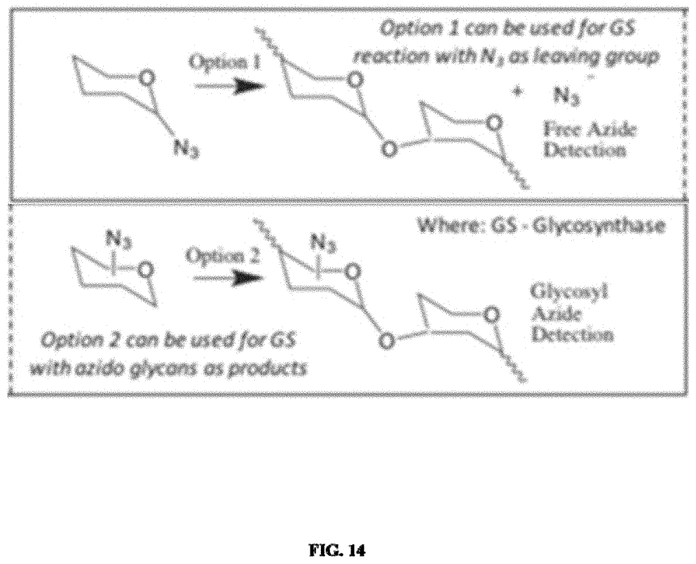

There are two possible strategies to monitor such a GS reaction: either by measuring the residual glycosyl azide donor sugar or the free azide produced (hydrazoic acid). It is possible to monitor the disappearance of glycosyl azide (lower fluorescence than empty vector control) or the increase in free azide concentration (higher fluorescence than control) depending on the reaction rate differences, sensitivity of the fluorophore to triazole moiety, washing steps to minimize background, and the concentration of the click compatible reagents (FIG. 14).

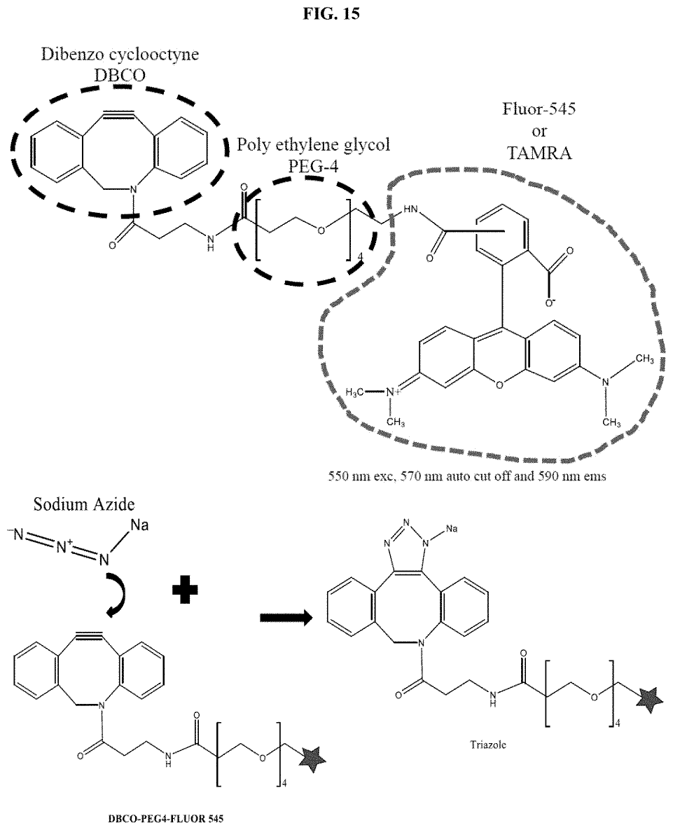

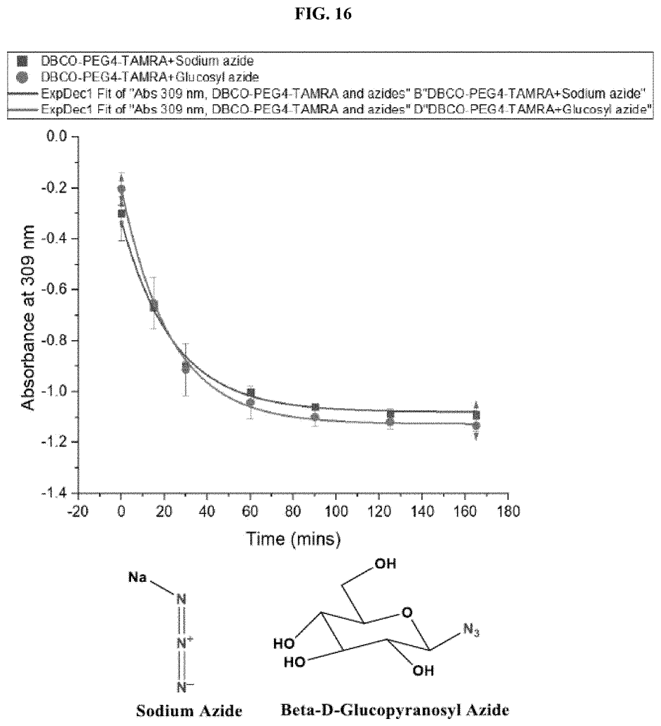

The strain-promoted azide-alkyne cycloaddition (SPAAC) reaction of either sodium azide (inorganic azide) or β-D-glucopyranosyl azide (organic azide) with DBCO-PEG4-Fluor 545 was studied in vitro (FIG. 15). The progress of the SPAAC reaction was quantitatively monitored by measuring the solution absorbance at 309 nm wavelength (λ₃₀₉), which is the characteristic wavelength for alkyne groups. A decay in the measured absorbance at λ₃₀₉ nm is indicative of the SPAAC reaction between the alkyne group in the dibenzocyclooctyne or DBCO moiety along with either the inorganic azide or organic azido groups (FIG. 16). The SPAAC reaction kinetics data was fitted to a simple exponential decay function to obtain the apparent rate constants for the formation of the triazole moiety. The rate constants for sodium azide (0.0412±0.006 s⁻¹) and glucosyl azide (0.043±0.003 s⁻¹) were similar, therefore the SPAAC reaction proceeds at comparable rates for both organic and inorganic azides.

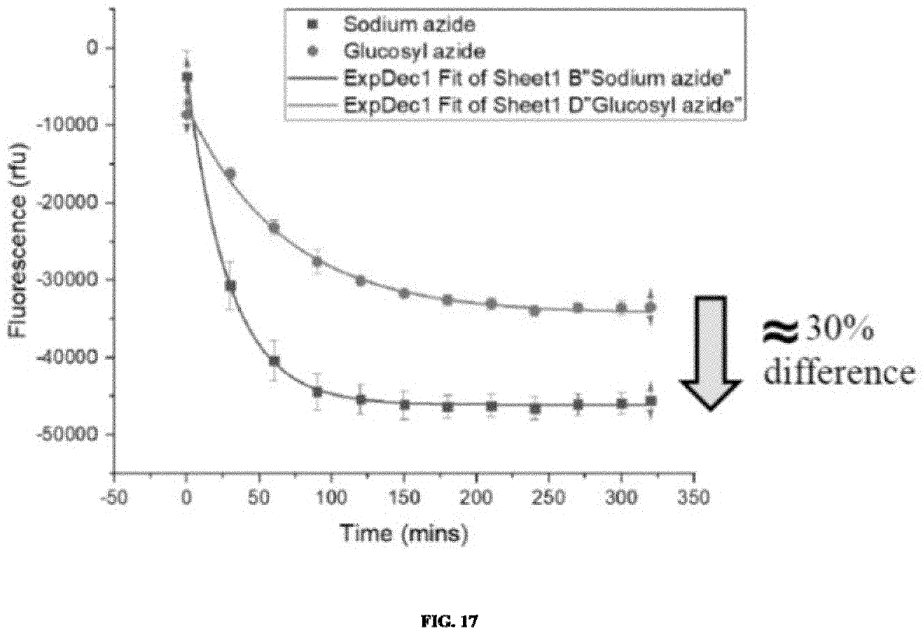

The solution fluorescence for each SPAAC reaction mixture was monitored at every time point in tandem with each absorbance measurement (FIG. 17). The excitation (550 nm) and emission (590 nm) wavelength filters used to quantify the red fluorescence measurements were specific to the Fluor545 fluorophore or standard tetramethylrhodamine (TAMRA) dye. The kinetic traces of the fluorescence data also indicated a sharp decrease in the solution fluorescence over a period of 30 minutes. This decrease in fluorescence is concomitant with the reduction in λ₃₀₉ nm absorbance seen during the SPAAC reaction, also corroborating that the reaction was complete within about 30 to 60 mins for both glucosyl azide and sodium azide. A difference of about 30% in the absolute fluorescence intensities of the corresponding triazole products formed during the SPAAC reaction of DBCO-PEG4-Fluor 545 with sodium and glucosyl azide, respectively, was observed. This absolute decrease in fluorescence intensity may be attributed to the currently poorly understood photophysical interactions of the TAMRA red dye fluorophore group to either the glycosylated versus non-glycosylated triazole moiety, formed during the SPAAC reaction, to observe differential quenching in observed red fluorescence.

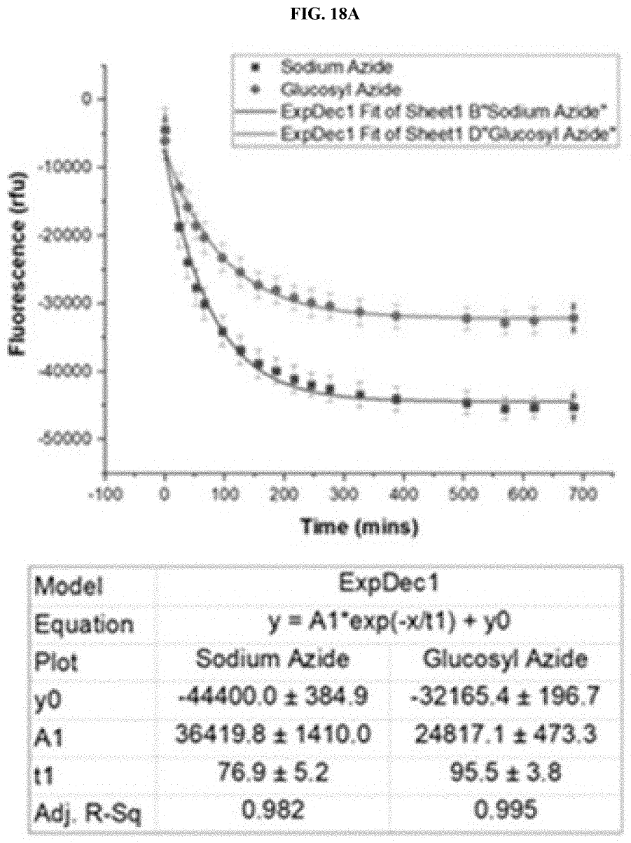

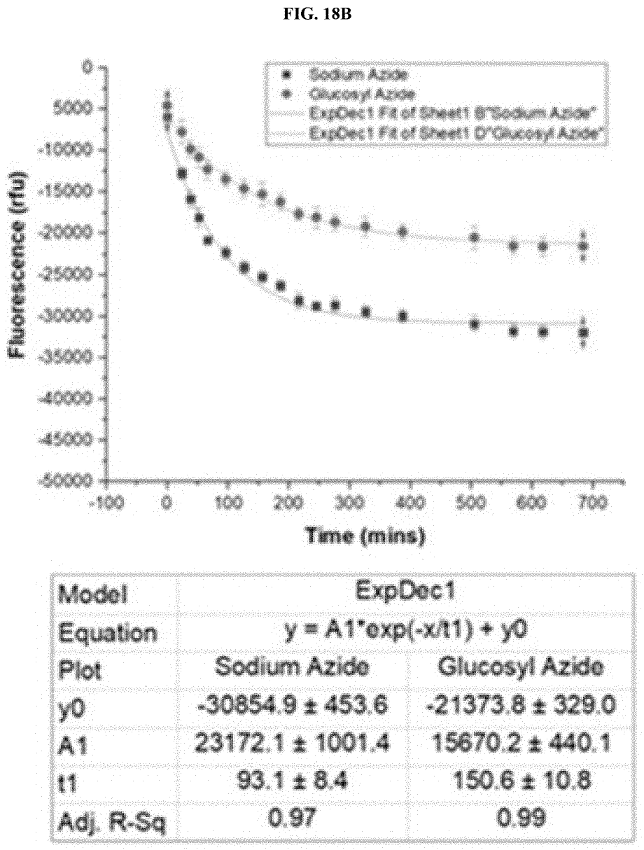

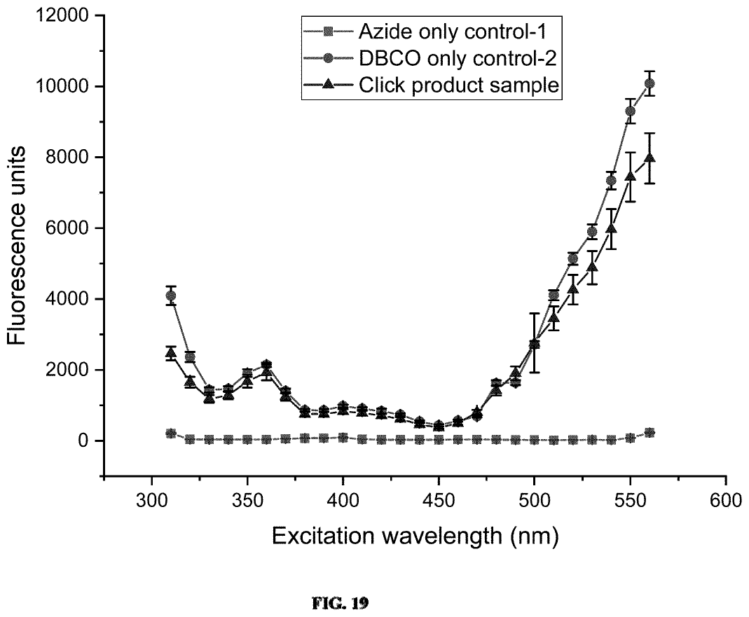

No significant impact of lower reaction temperatures on this differential fluorescence phenomenon was observed (FIGS. 18A-18B). Based on the full absorbance and excitation/emission fluorescence data, there seems to be a maximum decay in emission fluorescence for the SPAAC products, versus the unreacted DBCO-PEG4-Fluor 545 dye, only close to the 550 nm excitation wavelength for the Fluor545 moiety (FIG. 19). Nevertheless, even excitation at lower wavelengths (480-550 nm) still shows a slight differential decrease in the emission fluorescence for the SPAAC products formed with sodium azide versus glucosyl azide.

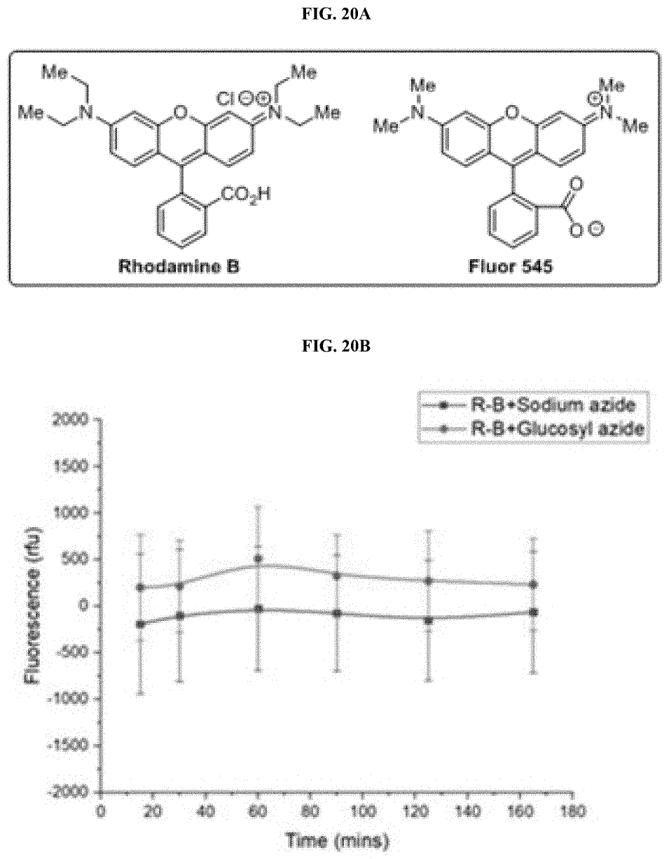

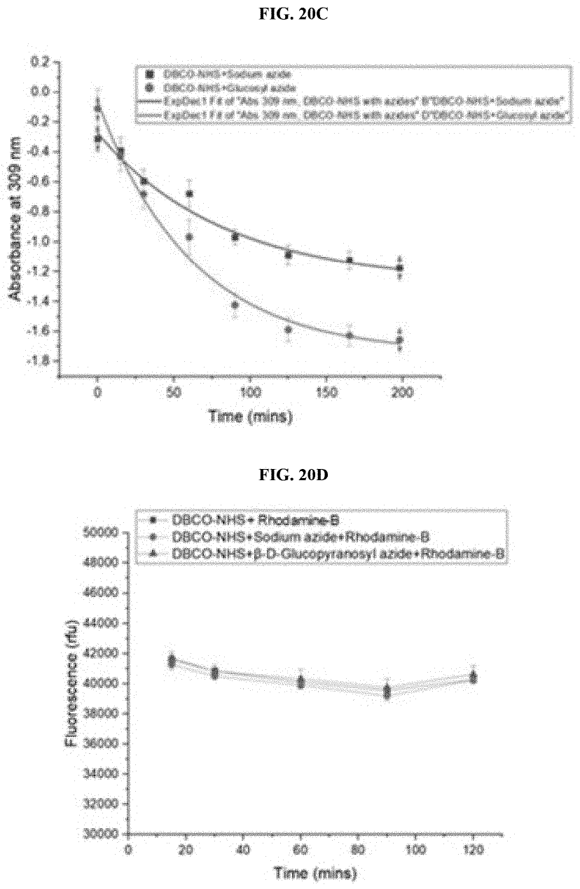

To assess the generality of the differential photophysical phenomenon observed with DBCO-PEG4-Fluor and organic/inorganic azides, studies were performed with organic and inorganic azides and a structural homolog of TMRA dye (e.g., Rhodamine-B). Fluor-545 (Tetramethyl rhodamine) and Rhodamine-B (Tetraethyl rhodamine) dyes are structurally similar with a minor difference as the methyl groups in Fluor-545 are replaced by ethyl groups in Rhodamine-B (FIG. 20A). However, the Fluor-545 dye moiety alone is not readily available from commercial sources. Commercially available derivatives are either tagged with esters, azides, or other functional groups that might interfere with the SPAAC reaction. Thus, the effect of free azide or SPAAC derived triazole products on the fluorescence of Rhodamine-B dye was instead examined to identify the effect on the fluorescence of the fluorophore moiety alone and the fluorophore moiety in the presence of exogenously added azides or pre-formed triazole products. This experiment was conducted to explore why the Fluor-545 moiety fluorescence differentially reduces upon completion of the SPAAC reaction for organic versus inorganic azides. No significant difference in the absolute fluorescence for Rhodamine-B dye in the presence of glucosyl-azide versus sodium azide was observed, suggesting that the triazole moiety is likely critical to observing any differences (FIG. 20B).

The potential influence of inter-molecular interactions of a glycosylated versus non-glycosylated triazole moiety with the Rhodamine-B dye on dye fluorescence was similarly examined. Here, the SPAAC reaction between a model DBCO-moiety lacking a fluorophore group (i.e., DBCO-NHS) and each respective azide substrate was performed to form a triazole product before addition of Rhodamine-B dye to each reaction. Triazole product formation was confirmed during the SPAAC reaction by observed changes in absorbance at 309 nm at various time points (FIG. 20C). The addition of Rhodamine-B to either the glycosylated versus non-glycosylated triazole SPAAC products showed no significant change in fluorescence compared to the Rhodamine-B dye added along with DBCO-NHS by itself (FIG. 20D). This result suggests that intra-molecular interactions of the red fluorophore group with the triazole moiety, facilitated by the connected PEGylated linker, are necessary for the appropriate photophysical interactions that result in differential fluorescence observed for glycosylated versus non-glycosylated triazole-fluorophore based SPAAC products. The PEG linker likely facilitates specific intramolecular interactions between the triazole moiety, after reaction of DBCO moiety with either the azido sugar or the free azide, and the FLUOR-545 group in well-defined molecular orientations to facilitate donor-acceptor interactions through intramolecular charge transfer (ICT) or fluorescence resonance energy transfer (FRET) type photophysical interactions.

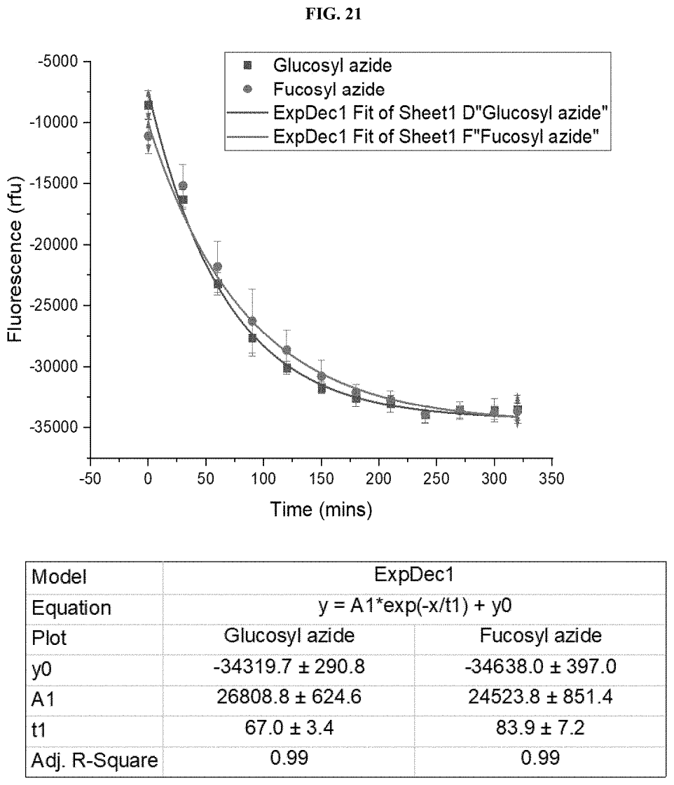

Changing the glycosyl moiety from glucose to fucose did not alter the relative trends in fluorescence patterns noted here (FIG. 21), suggesting that glycosylated triazoles behave similarly. This triazole moiety is clearly distinct from the one formed from an inorganic azide and hence that could explain why the sugar azides behave similarly based on the change in fluorescence associated with the Fluor 545 group.

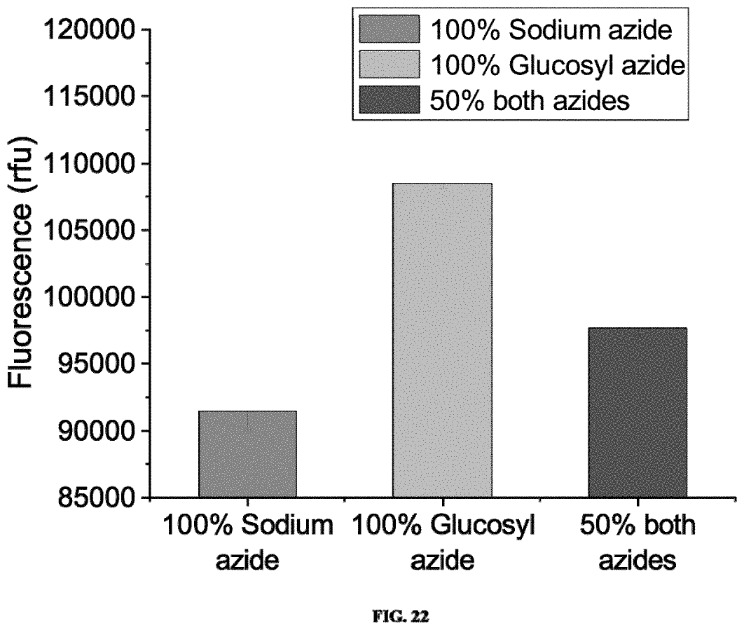

The potential to utilize the differential fluorescence of fucosyl triazoles and unsubstituted triazoles as a means to detect a varying range of substrate/product concentration limits (e.g., unreacted glycosyl-azide substrate versus free released azide products) by using the SPAAC reaction was examined in vitro (FIG. 22). The fluorescence of the SPAAC reaction with a combination of both azides was found to be at a mid-point between the results observed for sodium azide and β-D-glucopyranosyl independently. Thus, mixtures of organic and inorganic azides formed by glycosynthases of varying degrees of catalytic efficiency may remain differentiable using this SPAAC-fluorescence detection method.

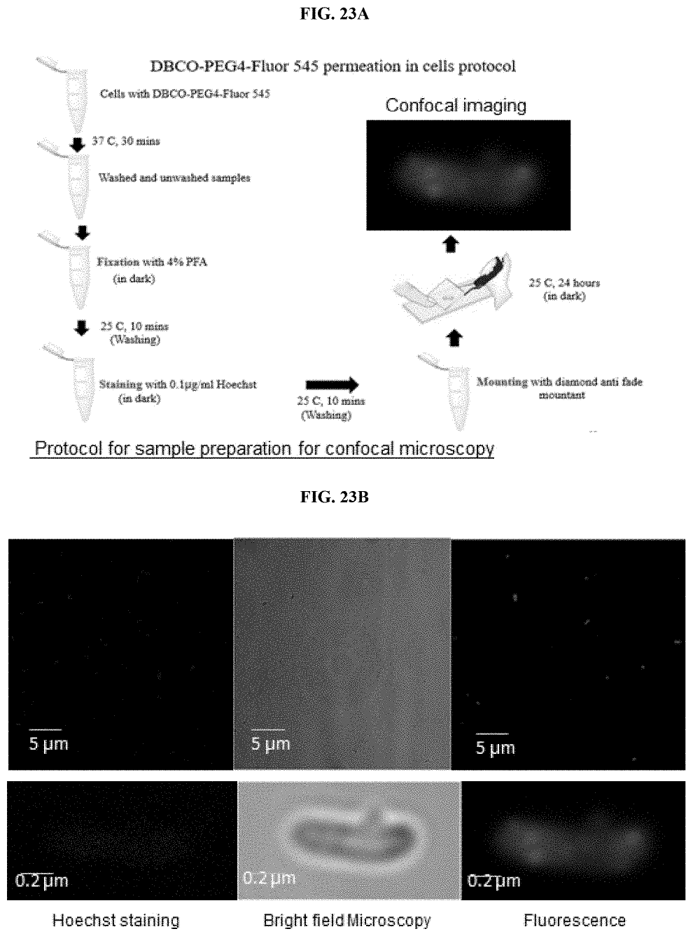

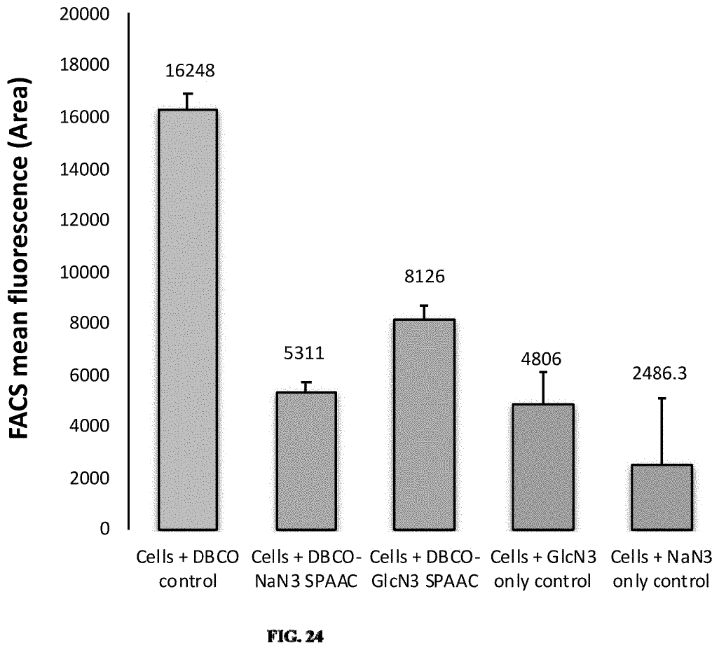

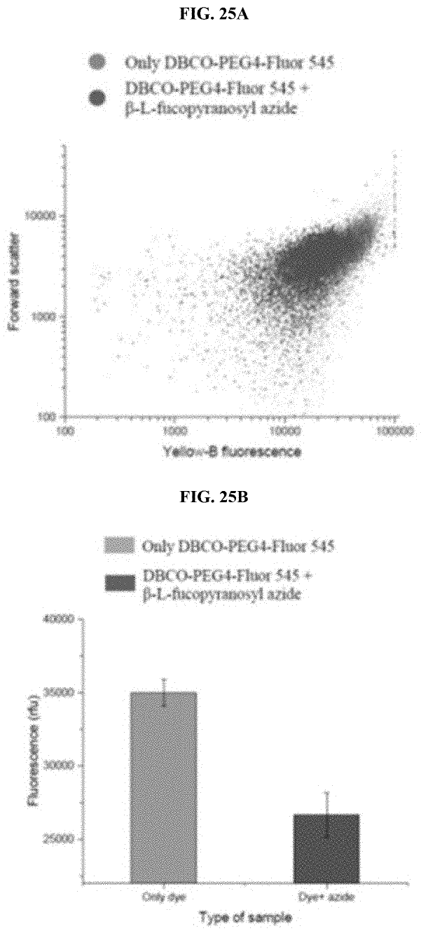

The SPAAC reaction using DBCO-PEG4-FLUOR 545 is further able to give a differential fluorescence response for GS products formed and/or unreacted substrates present under in-vivo conditions. Confocal fluorescence microscopy was performed to confirm that the fluorescent SPAAC reagent (i.e., DBCO-PEG4-FLUOR 545) could readily permeate inside E. coli cells (FIG. 23). Flow cytometry further corroborated the efficiency of permeation of the SPAAC reagents into the cells. Flow cytometry was utilized to determine total fluorescence intensity and distribution for E. coli cells containing SPAAC reaction products for DBCO-PEG4-FLUOR 545 reacted with either type of azide alone (FIG. 24). As observed in the in-vitro assays, E. coli cells containing SPAAC reaction products for glucosyl-azide provided a different fluorescence intensity than the cell population containing SPAAC reaction products for sodium azide alone.

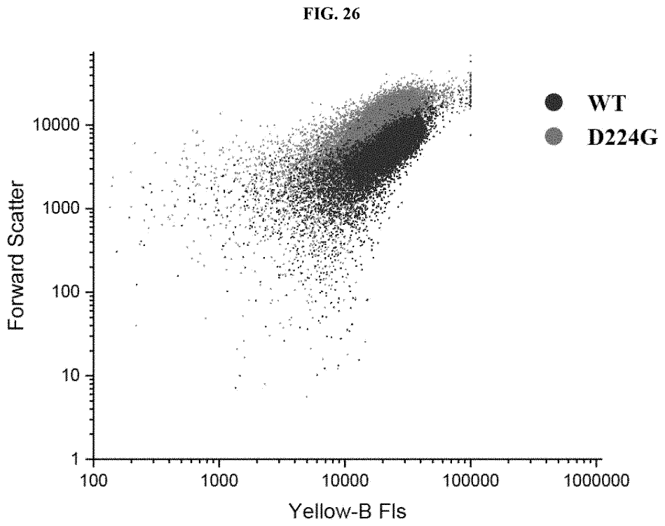

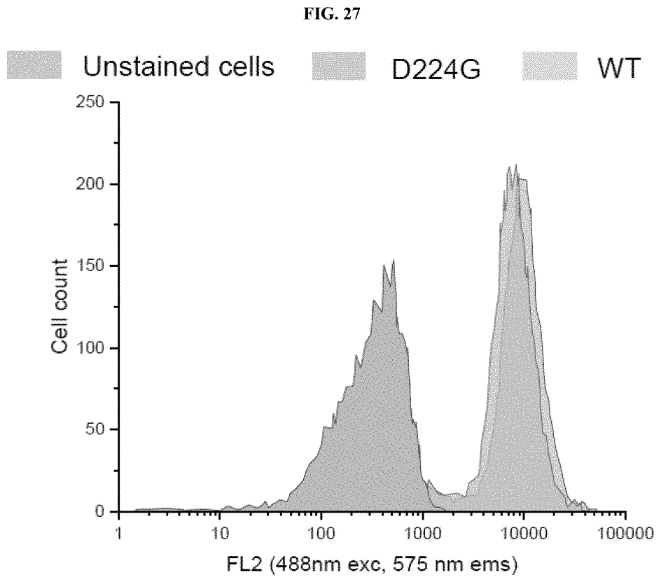

Flow cytometry (and FACS) confirmed that E. coli cells expressing D224G provided a distinguishable decrease in fluorescence intensity compared to TmAfc wild type GH after conducting the GS and SPAAC reaction sequence (FIG. 26 and FIG. 27).

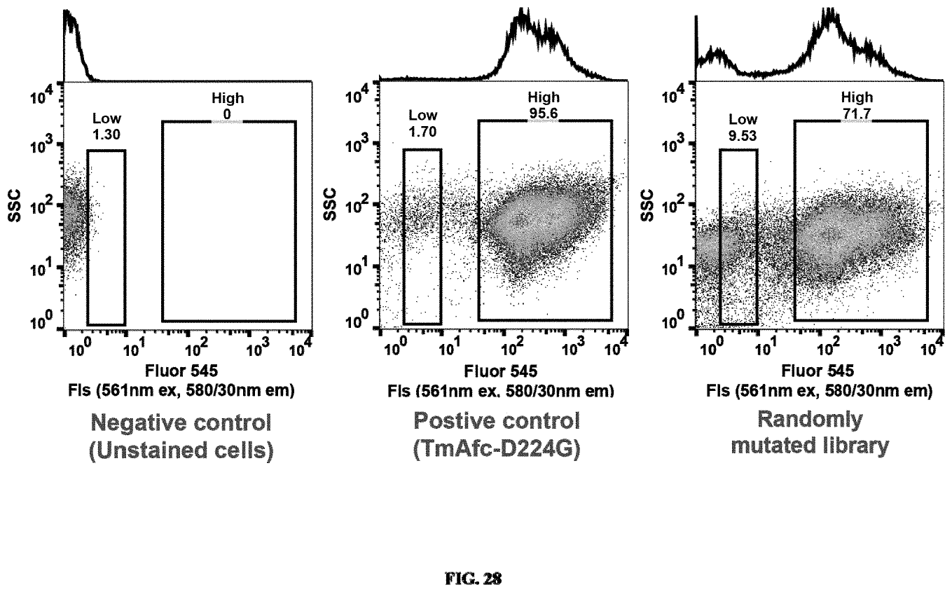

While in the case of the Fluor 545 fluorophore, either 488 nm or 561 nm laser lines can be used based on availability of suitable instrumentation capabilities, however, an improved signal-to-noise ratio is clearly observed for the latter excitation wavelength for sorting GS mutants (FIG. 28).

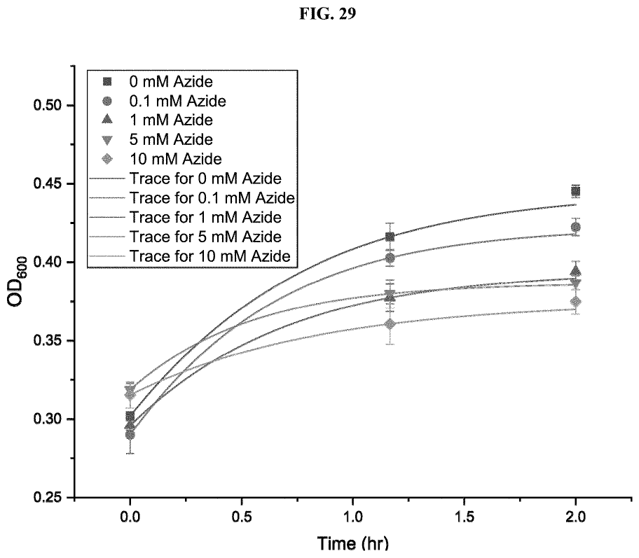

Currently, no major toxicity is observed as a result of inorganic azide generation at the substrate concentrations utilized herein (FIG. 29). Overall, these proof-of-concept uHTS methodology development results suggest that it would be possible to sort mutant GSs with increased activity based on decreased fluorescence expected for the SPAAC reaction products in vivo.

### Example 4: Determine Mutations Needed to Improve GS Activity

Once the baseline FACS method is established, this method can be used to screen a large library of GS mutants prepared by various methods. In certain embodiments, a nucleophile site saturation mutagenesis library (10²-10³ clones screened) can be created to search for other possible nucleophile mutants with GS activity. This experiment can also validate the HTS assay if one can identify whether the D242S vs. D242G SsFucA GS mutant gives higher catalytic activity with fucosyl azides. In certain embodiments, a random mutagenesis library (-10³-10⁶ clones) introduces an average of 2-4 mutations per gene using error prone PCR to search for mutations that can increase the catalytic activity for the target GS. Primary screening of cloned cells is carried out using FACS to identify about 50-100 clones for detailed secondary screening using a microplate based assay for a quantitative estimation of the GS activity.

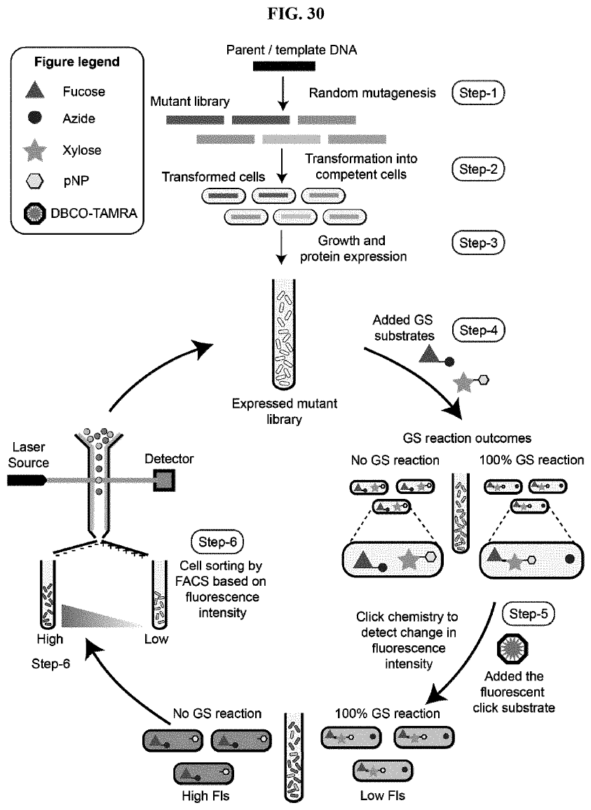

In certain embodiments this approach has been exemplified (FIG. 30). Random mutagenesis was performed using TmAfc-D224G as a template (control) and the epPCR mutant library was sorted in a FACS cell sorter (using the 488 nm excitation laser filter) to identify mutants with increased fucosynthase activity towards pNP-xylose as an acceptor after two rounds of FACS sorting. The average number of mutations introduced during epPCR was about 3 to 4 mutations per mutant construct.

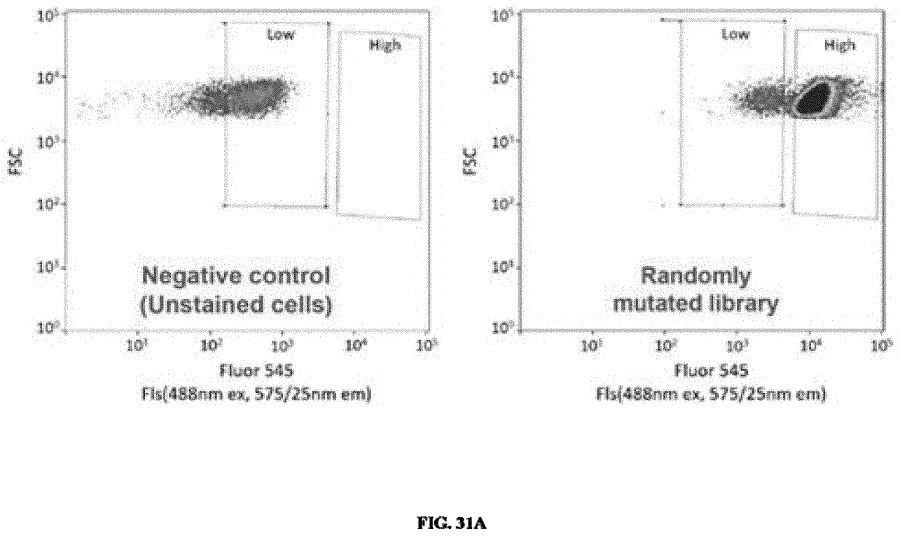

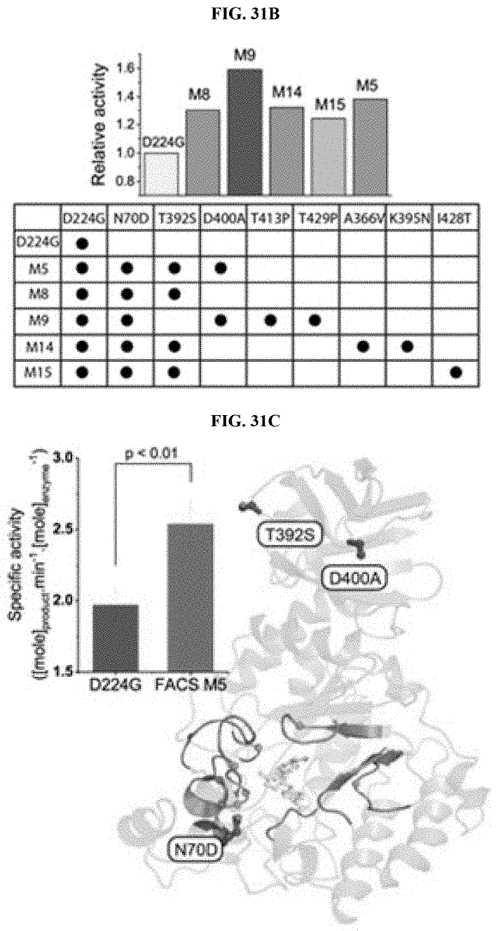

The fluorescence intensities of unstained E. coli cells (negative control) and cells expressing template D224G protein (positive control) were first captured to optimize the FACS instrument parameters (e.g., pressure, gain) and build the fluorescence gates for sorting. Two distinct populations with fluorescence intensities with ranges differing over an order of magnitude were clearly observed when the epPCR mutant library cells were analyzed using FACS (FIG. 31A).

This differential change in fluorescence is closely dependent on the excitation wavelength laser available (e.g., 488 nm blue vs. 561 nm yellow lasers). Two fluorescence gates, differing over <10-fold magnitude, referred to as “Low” and “High”, were used with the 488 nm laser filter to identify these cell populations. However, it is indeed possible to modify the signal-to-noise to further increase sorting efficiency when using the 561 nm laser filter to select fluorescence gates that could differ over >10-fold magnitude to identify and classify these cell populations (FIG. 28).

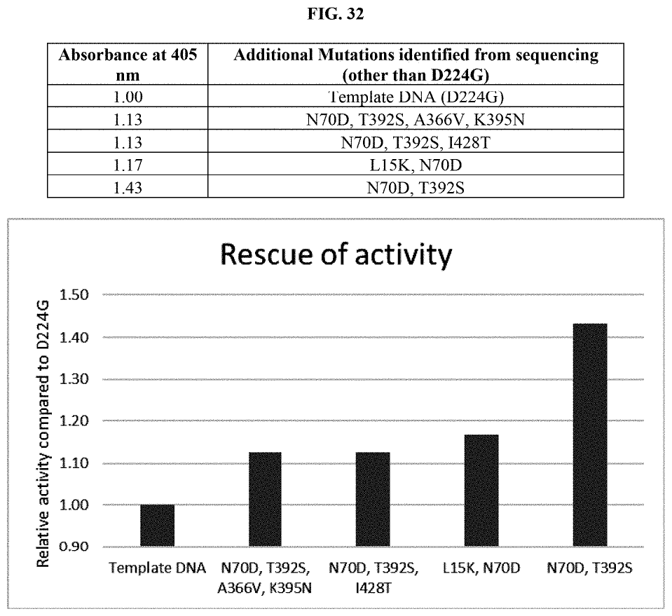

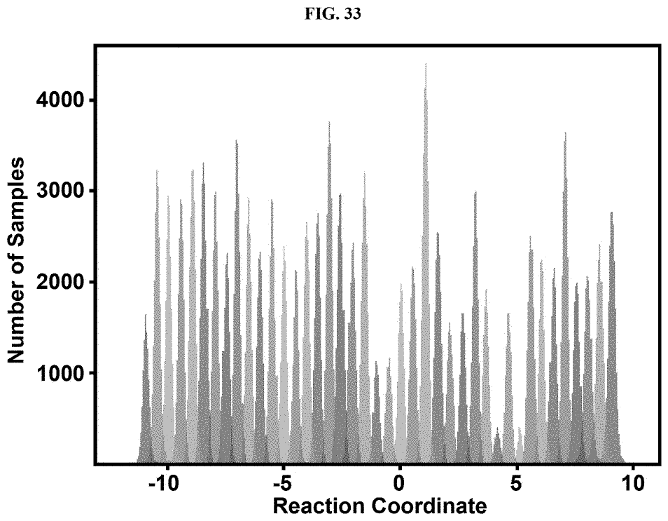

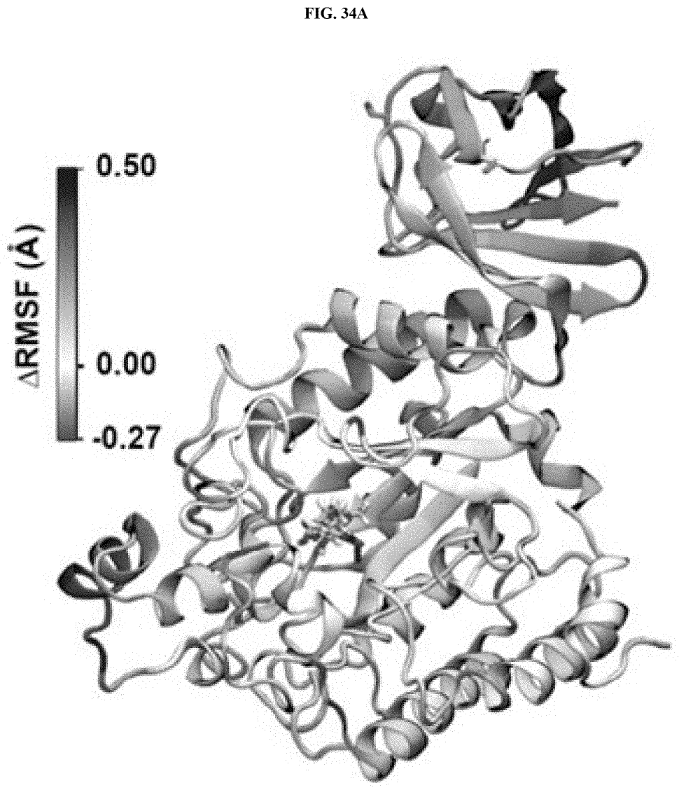

Cells which had fluorescence in the Low gate for the epPCR mutant library, were separated by FACS (using 488 nm blue laser) and collected into a single tube containing LB recovery/growth media. The first round sorted cells were regrown and then subjected to a second round of FACS (using 488 nm blue laser again) sorting to minimize chances of isolating any potential false positives collected in the first round. During the second round of sorting, individual cells in the Low gate were similarly sorted but now collected as individual cells in a 96-well plate with LB recovery/growth media for further characterization. The individually sorted cells were then grown, and protein expression was induced in a 96-well culture plate. After protein expression, the cells were lysed and the pNP-fucose substrate along with the external nucleophile sodium azide was added to check for expressed enzyme chemical rescue activity as a secondary screen prior to conducting detailed DNA sequencing for top performing mutants from this secondary screen. The FACS (using 488 nm blue laser) sorted single-cell epPCR mutants with improved chemical rescue activity compared to the template D224G control (FIG. 31B and FIG. 32). Top hits in the chemical rescue activity screen were then selected for plasmid DNA extraction and subsequent DNA sequencing of identified positive GS mutants. Unique mutants identified after DNA sequencing were individually expressed and purified proteins were used to perform in-vitro glycosynthase reactions. From a simple two round sorting analysis, at least five unique fucosynthase mutants that give 1.3-1.6 fold higher glycosynthase activity have been identified (FIG. 31C). N70D was the most highly conserved mutation identified for all active mutants of the parent WT construct (SEQ ID NO:1), which in combination with T392S and D224G mutations gave significantly higher fucosynthase activity compared to the original template (D224G) (SEQ ID NO:2).

One of these mutants (M5, SEQ ID NO:6) carrying three new mutations, in addition to D224G (or TmAfc-D224G-N70D-T392S-D400A), was expressed and purified by SDS-PAGE to conduct systematic in-vitro enzyme activity assays (FIG. 12). The identity of both the GS reaction products that were first qualitatively characterized using TLC were then quantitatively analyzed using HPLC-UV analysis. The specific activities of the M5 mutant, determined from the initial rate of the glycosynthase reaction between P-L-fucopyranosyl azide and pNP-β-D-xylopyranoside, was 29% greater than the template D224G (FIG. 31C). These results are also consistent with the ˜1.3 fold increased chemical rescue activity reported for M5 (FIG. 4B). Here, wild-type fucosidase (with intact nucleophile at D224) gave no measurable glycosynthase activity and therefore fucosynthase activity data is not shown here for this construct. Mutation of the asparagine residue (N70D) was the most highly conserved residue near the active site for nearly all identified mutants with improved fucosynthase activity. It is possible that N70D mutation could alter the interaction of neighboring Trp residue and helps with improved docking of the substrate in the active site (FIG. 31C).

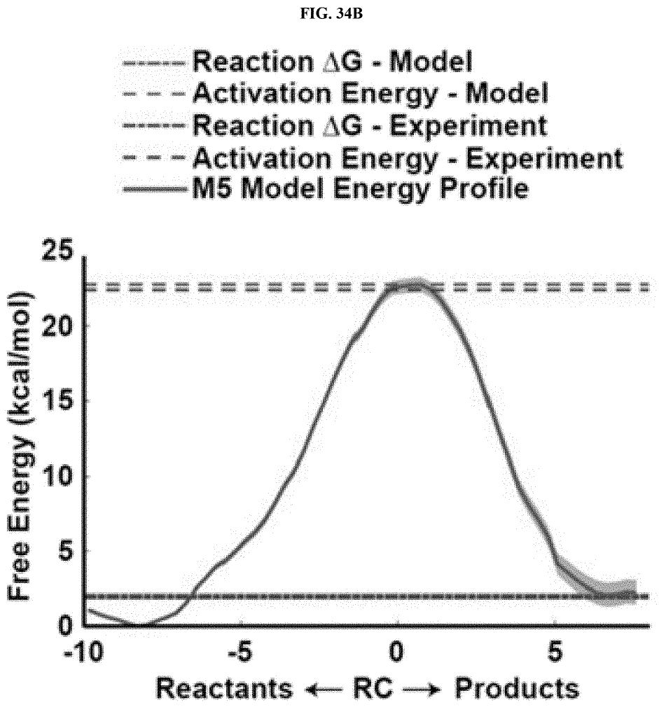

The M5 mutant construct was modeled and simulated to investigate structural features behind the improved glycosynthetic activity compared to the D224G single mutant. Unbiased molecular mechanics (MM) simulations were used to characterize the structural and dynamic changes associated with the mutations (FIG. 34A) and hybrid quantum mechanics/molecular mechanics (QM/MM) simulations were used to obtain the free energy profile of the α(1,4) glycosynthetic reaction within the enzyme active site (FIG. 34B). The three additional mutations in the M5 construct did not greatly change the protein conformation but did result in a decrease in the rigidity of almost all parts of the enzyme (FIG. 34A). This result suggests that the means by which the M5 mutant improves upon the glycosynthetic activity of the single-mutant is by loosening the highly specific structure of the wild-type active site (which would have evolved to suit glycoside hydrolysis exactly) in favor of a more general α-fucosyl oligosaccharide binding site with a de-emphasized preference for hydrolysis over synthesis.

The QM/MM simulations were in close agreement with the experimental results, both in terms of activation energy and overall reaction ΔG (FIG. 34B). This close agreement strongly suggests that the reaction step is rate-limiting in turnover of this enzyme, in which case the mechanism of increased activity in the M5 construct over the single mutant would be a reduction in the forward reaction activation energy barrier, albeit one too small to confidently distinguish with this computational model (based on the experimental results, the activation energy should change by just 0.15 kcal/mol at ˜333 K). Taken in the context of the MM results, the most likely explanation for the improved activity is a subtle loosening in the tightness of the active site in such a way as to better permit the glycosynthetic transition state, perhaps by making additional room for the slightly longer C—N bond in the glycosynthase over the shorter C—O bond in the wild-type enzyme.

### Example 5: Determination of Mutations Needed to Alter Substrate Specificity

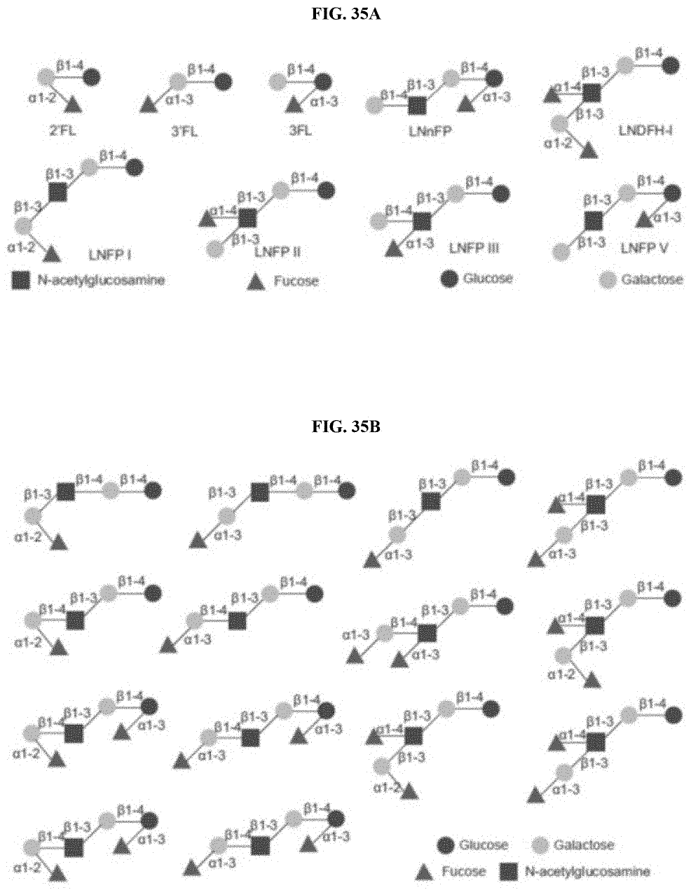

One can explore the depth of potential for selected enzymes to synthesize multiple types of fucosylated oligosaccharides. In one aspect, one can engineer enzymes that can produce a broad range of oligosaccharides, which can then be tested for beneficial activities, such as effective antimicrobial and antibiofilm agents. At present, synthesis methods are available for some simple fucosylated oligosaccharides, including 2′FL; 3′FL; 3FL; Lacto-N-fucopentaose II, III, and V (LNFP-I, -II, -III, and -V); Lacto-N-neofucopentaose (LNnFP), and Lacto difucohexaose I (LNDFH-I) (FIG. 35A). One can focus on engineering GS enzymes to create novel fucosylated oligosaccharides that are currently more challenging to produce commercially (FIG. 35B).

To achieve this goal, one can use molecular models of fucosynthases to determine which changes to binding sites are required (if any) to allow for effective binding of alternate acceptor oligosaccharides, still employing a β-glycosyl azide donor molecule. This work can involve a feedback loop between the computational model and wet-lab testing of the resulting predictions. Specifically, in addition to the focus on active site engineering, one can characterize the binding site residues and determine correlations that distinguish between residue identity and binding affinity. Molecular models for enzymes can be adopted from. crystal structures of the enzyme in question or from a closely related GH29 for which a crystal structure is available. To predict whether particular mutations are advantageous for binding alternate substrates, one can model homologous (GH29) chemical transformations from successffilly bound substrates to the desired substrates, and use the resulting data on specific substrate-residue interactions to determine which mutations would be advantageous. Creating these binding-site mutants and analyzing their activity can test these predictions.

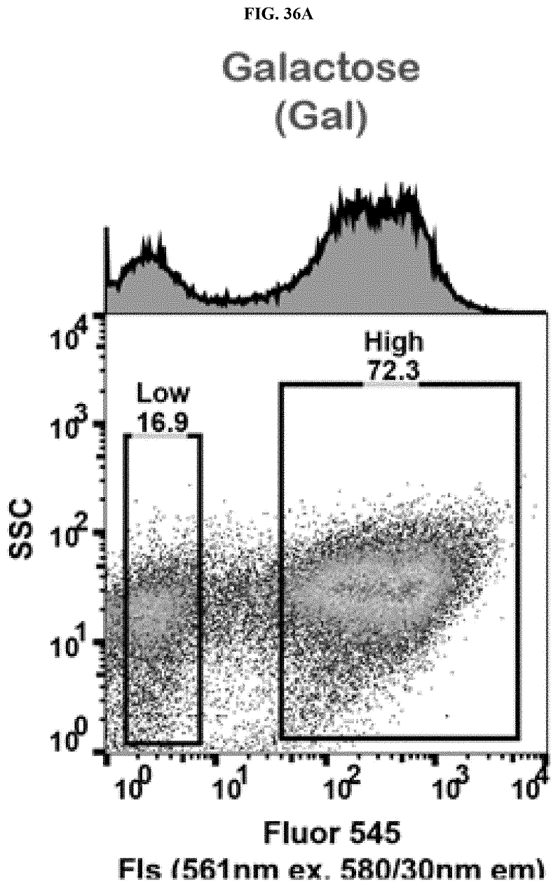

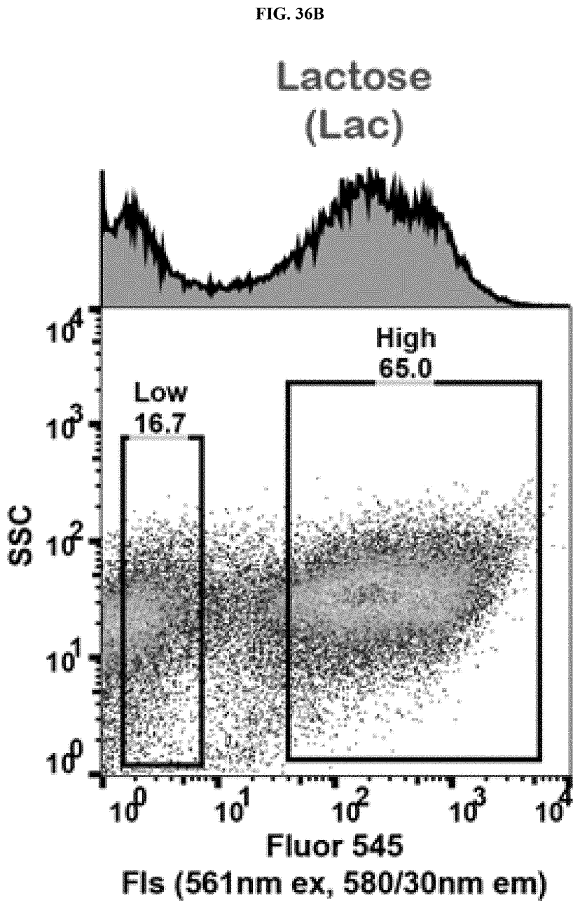

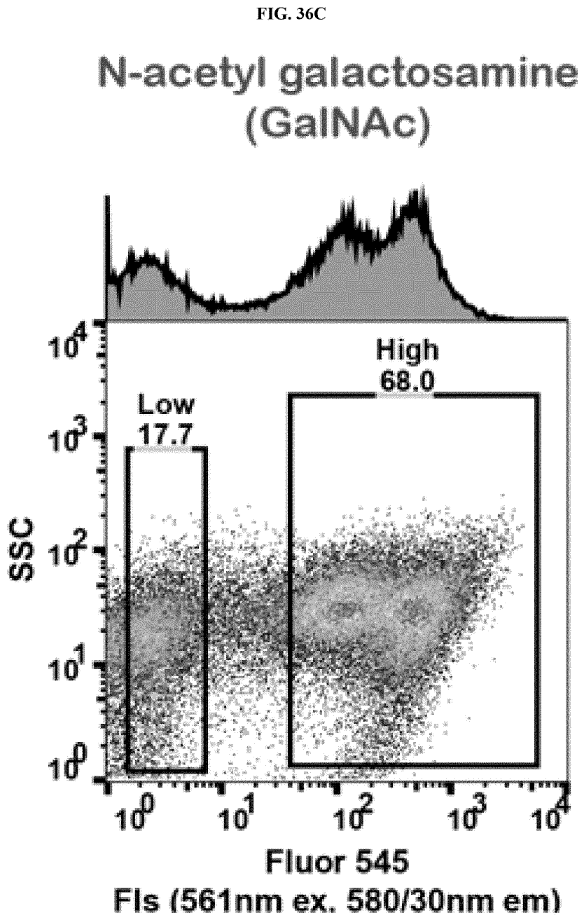

An uHTS strategy has also been employed to sort a mutant D224G epPCR library to further identify novel GSs with altered substrate specificity by changing the acceptor sugar from pNP-xylose to either lactose, N-acetylglucosamine, or galactose (FIG. 36). Here, using a more sensitive 561 nm based FACS excitation laser for cell sorting, a nearly 10-fold increase in the relative percentage of mutant cells identified in the low gate (˜17% of total population) compared to the starting D224G control (1.7% of total population) is observed (FIG. 28). These results clearly highlight this approach is readily adapted to evolve and screen GS activity for novel acceptor sugars as well.

Sequence Listings:
WT
SEQ ID NO: 1
MISMKPRYKPDWESLREHTVPKWFDKAKFGIFIHWGIYSVPGWATPTGELGKVPMDAWFFQNPY
AEWYENSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNDMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPTGEKIVIEDLNLSAGTVRHFLTGERLSFKNVGKNLEITVPKKLLETDSITLVLEAVE
E
D224G (Template DNA)
SEQ ID NO: 2
MISMKPRYKPDWESLREHTVPKWFDKAKFGIFIHWGIYSVPGWATPTGELGKVPMDAWFFQNPY
AEWYENSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPTGEKIVIEDLNLSAGTVRHFLTGERLSFKNVGKNLEITVPKKLLETDSITLVLEAVE
E
D224G-N70D-L15K
SEQ ID NO: 3
MISMKPRYKPDWESKREHTVPKWFDKAKFGIFIHWGIYSVPGWATPTGELGKVPMDAWFFQNPY
AEWYEDSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPTGEKIVIEDLNLSAGTVRHFLTGERLSFKNVGKNLEITVPKKLLETDSITLVLEAVE
E
D224G-N70D-T3925
SEQ ID NO: 4
MISMKPRYKPDWESLREHTVPKWFDKAKFGIFIHWGIYSVPGWATPTGELGKVPMDAWFFQNPY
AEWYEDSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPSGEKIVIEDLNLSAGTVRHFLTGERLSFKNVGKNLEITVPKKLLETDSITLVLEAVE
E
D224G-N70D-T3925-A366V-K395N
SEQ ID NO: 5
MISMKPRYKPDWESLREHTVPKWFDKAKFGIFIHWGIYSVPGWATPTGELGKVPMDAWFFQNPY
AEWYEDSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCVKTEDGTEIRFTRKCNRIF
VIFLGIPSGENIVIEDLNLSAGTVRHFLTGERLSFKNVGKNLEITVPKKLLETDSITLVLEAVE
E
D224G-N70D-T3925-D400A
SEQ ID NO: 6
MISMKPRYKPDWESLREHTVPKWFDKAKFGIFIHWGIYSVPGWATPTGELGKVPMDAWFFQNPY
AEWYEDSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPSGEKIVIEALNLSAGTVRHFLTGERLSFKNVGKNLEITVPKKLLETDSITLVLEAVE
E
D224G-N70D-T3925-1428T
SEQ ID NO: 7
MISMKPRYKPDWESLREHTVPKWFDKAKFGIFIHWGIYSVPGWATPTGELGKVPMDAWFFQNPY
AEWYEDSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPSGEKIVIEDLNLSAGTVRHFLTGERLSFKNVGKNLETTVPKKLLETDSITLVLEAVE
E
D224G-N70D-D400A-T413P-T429P
SEQ ID NO: 8
MISMKPRYKPDWESLREHTVPKWFDKAKFGIFIHWGIYSVPGWATPTGELGKVPMDAWFFQNPY
AEWYEDSLRIKESPTWEYHVKTYGENFEYEKFADLFTAEKWDPQEWADLFKKAGAKYVIPTTKH
HDGFCLWGTKYTDFNSVKRGPKRDLVGDLAKAVREAGLRFGVYYSGGLDWRFTTEPIRYPEDLS
YIRPNTYEYADYAYKQVMELVDLYLPDVLWNGMGWPEKGKEDLKYLFAYYYNKHPEGSVNDRWG
VPHWDFKTAEYHVNYPGDLPGYKWEFTRGIGLSFGYNRNEGPEHMLSVEQLVYTLVDVVSKGGN
LLLNVGPKGDGTIPDLQKERLLGLGEWLRKYGDAIYGTSVWERCCAKTEDGTEIRFTRKCNRIF
VIFLGIPTGEKIVIEALNLSAGTVRHFLPGERLSFKNVGKNLEIPVPKKLLETDSITLVLEAVE
E

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed:

1. A method of determining if a protein has transglycosylase activity, the method comprising:
contacting the protein with an azido glycosyl donor and a glycosyl acceptor to form a system, and
measuring any change in azide concentration in the system.

2. The method of claim 1, wherein the azido glycosyl donor is substituted with an azido group at an anomeric or non-anomeric carbon.

3. The method of claim 1, wherein the azide concentration of the system comprises an inorganic azide and an anomeric glycosyl azide species or a non-anomeric glycosyl azide species.

4. The method of claim 1, wherein the measuring step comprises contacting the system with a reagent comprising a strained alkyne coupled to a dye, under conditions that allow for reaction of the strained alkyne with any azide or azido compound present in the system.

5. The method of claim 4, wherein the reagent comprises bicyclo[6.1.0]nonyne (BCN), dibenzocyclooctyne (DBCO), or any other strained alkyne.

6. The method of claim 4, wherein the reagent comprises 5-carboxytetramethylrhodamine (5-TAMRA), 6-carboxytetramethylrhodamine (6-TAMRA), or any combinations thereof.

7. The method of claim 4, wherein the strained alkyne and the dye are covalently linked by a linker in the reagent.

8. The method of claim 7, wherein the linker comprises a polyethylene glycol linker.

9. The method of claim 1, wherein the measuring step uses as a control a protein that has no measurable transglycosylase activity or has a known transglycosylase activity.

10. The method of claim 1, wherein the protein is a mutated glycosyl hydrolase (GH).

11. The method of claim 1, wherein the protein is expressed in a cell.

12. The method of claim 11, wherein the cell comprises E. coli or Pichia pastoris.

13. The method of claim 11, wherein the system is within the cell (intracellular).

14. The method of claim 4, wherein the measuring step comprises monitoring fluorescence of the system.

15. The method of claim 13, wherein fluorescence activated cell sorting (FACS) is used to separate individual cells by measured fluorescence.

16. The method of claim 15, which is configured for high-throughput screening.

17. A polypeptide comprising an amino acid sequence of SEQ ID NO:1,
wherein the polypeptide comprises the mutation D224G (SEQ ID NO:2) with respect to SEQ ID NO:1,
wherein the polypeptide further comprises at least one additional mutation selected from the group consisting of L15K, N70D, A366V, T392S, K395N, D400A, T413P, I428T, and T429P.

18. The polypeptide of claim 17, wherein the at least one additional mutation to an amino acid sequence of SEQ ID NO:2 is selected from the group consisting of: L15K-N70D; N70D-T392S; N70D-T392S-A366V-K395N; N70D-T392S-D400A; N70D-T392S-I428T; N70D-D400A-T413P-T429P.

19. The polypeptide of claim 18, which is selected from the group consisting of SEQ ID NOs:3-8.


SEQUENCE LISTING (18 sequences)

SEQ ID NO: 1 - protein, 449 aa, Artificial Sequence
  TmAfc
  WT
MISMKPRYKPDWESLREHTVPKWFDKAKFGIFIHWGIYSVPGWATPTGELGKVPMDAWFFQNPYAEWYENSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNDMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPTGEKIVIEDLNLSAGTVRHFLTGERLSFKNVGKNLEITVPKKLLETDSITLVLEAVEE

SEQ ID NO: 2 - protein, 449 aa, Artificial Sequence
  TmAfc
  D224G
MISMKPRYKPDWESLREHTVPKWFDKAKFGIFIHWGIYSVPGWATPTGELGKVPMDAWFFQNPYAEWYENSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPTGEKIVIEDLNLSAGTVRHFLTGERLSFKNVGKNLEITVPKKLLETDSITLVLEAVEE

SEQ ID NO: 3 - protein, 449 aa, Artificial Sequence
  TmAfc
  D224G-N70D-L15K
MISMKPRYKPDWESKREHTVPKWFDKAKFGIFIHWGIYSVPGWATPTGELGKVPMDAWFFQNPYAEWYEDSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPTGEKIVIEDLNLSAGTVRHFLTGERLSFKNVGKNLEITVPKKLLETDSITLVLEAVEE

SEQ ID NO: 4 - protein, 449 aa, Artificial Sequence
  TmAfc
  D224G-N70D-T392S
MISMKPRYKPDWESLREHTVPKWFDKAKFGIFIHWGIYSVPGWATPTGELGKVPMDAWFFQNPYAEWYEDSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPSGEKIVIEDLNLSAGTVRHFLTGERLSFKNVGKNLEITVPKKLLETDSITLVLEAVEE

SEQ ID NO: 5 - protein, 449 aa, Artificial Sequence
  TmAfc
  D224G-N70D-T392S-A366V-K395N
MISMKPRYKPDWESLREHTVPKWFDKAKFGIFIHWGIYSVPGWATPTGELGKVPMDAWFFQNPYAEWYEDSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCVKTEDGTEIRFTRKCNRIFVIFLGIPSGENIVIEDLNLSAGTVRHFLTGERLSFKNVGKNLEITVPKKLLETDSITLVLEAVEE

SEQ ID NO: 6 - protein, 449 aa, Artificial Sequence
  TmAfc
  D224G-N70D-T392S-D400A
MISMKPRYKPDWESLREHTVPKWFDKAKFGIFIHWGIYSVPGWATPTGELGKVPMDAWFFQNPYAEWYEDSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPSGEKIVIEALNLSAGTVRHFLTGERLSFKNVGKNLEITVPKKLLETDSITLVLEAVEE

SEQ ID NO: 7 - protein, 449 aa, Artificial Sequence
  TmAfc
  D224G-N70D-T392S-I428T
MISMKPRYKPDWESLREHTVPKWFDKAKFGIFIHWGIYSVPGWATPTGELGKVPMDAWFFQNPYAEWYEDSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPSGEKIVIEDLNLSAGTVRHFLTGERLSFKNVGKNLETTVPKKLLETDSITLVLEAVEE

SEQ ID NO: 8 - protein, 449 aa, Artificial Sequence
  TmAfc
  D224G-N70D-D400A-T413P-T429P
MISMKPRYKPDWESLREHTVPKWFDKAKFGIFIHWGIYSVPGWATPTGELGKVPMDAWFFQNPYAEWYEDSLRIKESPTWEYHVKTYGENFEYEKFADLFTAEKWDPQEWADLFKKAGAKYVIPTTKHHDGFCLWGTKYTDFNSVKRGPKRDLVGDLAKAVREAGLRFGVYYSGGLDWRFTTEPIRYPEDLSYIRPNTYEYADYAYKQVMELVDLYLPDVLWNGMGWPEKGKEDLKYLFAYYYNKHPEGSVNDRWGVPHWDFKTAEYHVNYPGDLPGYKWEFTRGIGLSFGYNRNEGPEHMLSVEQLVYTLVDVVSKGGNLLLNVGPKGDGTIPDLQKERLLGLGEWLRKYGDAIYGTSVWERCCAKTEDGTEIRFTRKCNRIFVIFLGIPTGEKIVIEALNLSAGTVRHFLPGERLSFKNVGKNLEIPVPKKLLETDSITLVLEAVEE

SEQ ID NO: 9 - DNA, 33 bp, Artificial Sequence
  Primer
  Tm0306_D224A_Forward
gatgttctgtggaacgccatgggttggccggag

SEQ ID NO: 10 - DNA, 33 bp, Artificial Sequence
  Primer
  Tm0306_D224A_Reverse
ctccggccaacccatggcgttccacagaacatc

SEQ ID NO: 11 - DNA, 33 bp, Artificial Sequence
  Primer
  Tm0306_D224S_Forward
gatgttctgtggaactccatgggttggccggag

SEQ ID NO: 12 - DNA, 33 bp, Artificial Sequence
  Primer
  Tm0306_D224S_Reverse
ctccggccaacccatggagttccacagaacatc

SEQ ID NO: 13 - DNA, 33 bp, Artificial Sequence
  Primer
  Tm0306_D224G_Forward
gatgttctgtggaacggcatgggttggccggag

SEQ ID NO: 14 - DNA, 33 bp, Artificial Sequence
  Primer
  Tm0306_D224G_Reverse
ctccggccaacccatgccgttccacagaacatc

SEQ ID NO: 15 - DNA, 24 bp, Artificial Sequence
  Primer
  Tm0306_WT_epPCR_Vector_Forward
gaataaggatcctctagagtcgac

SEQ ID NO: 16 - DNA, 16 bp, Artificial Sequence
  Primer
  Tm0306_WT_epPCR_Vector_Reverse
catggcgatcgcctgg

SEQ ID NO: 17 - DNA, 16 bp, Artificial Sequence
  Primer
  Tm0306_WT_epPCR_Insert_Forward
ccaggcgatcgccatg

SEQ ID NO: 18 - DNA, 24 bp, Artificial Sequence
  Primer
  Tm0306_WT_epPCR_Insert_Reverse
gtcgactctagaggatccttattc